SEQUENCE LISTING (15 sequences)

SEQ ID NO: 1 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 1467 aa, Artificial Sequence
  Synthetic construct G2 (scF2)
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 3 - protein, 1511 aa, Artificial Sequence
  Synthetic construct G3 (scF3)
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 4 - protein, 1581 aa, Artificial Sequence
  Synthetic construct G4 (scF4)
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 5 - protein, 1644 aa, Artificial Sequence
  Synthetic construct G6 (scF5)
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 6 - protein, 1587 aa, Artificial Sequence
  Synthetic construct G4_flex (scF6)
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSQNPPVTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 7 - protein, 1644 aa, Artificial Sequence; identical to SEQ ID NO: 5
  Synthetic construct G6_opt (scF7)

SEQ ID NO: 8 - protein, 1581 aa, Artificial Sequence
  Synthetic construct G4_B3
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKCQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 9 - DNA, 4404 bp, Artificial Sequence
  Synthetic construct G2
gccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 10 - DNA, 4536 bp, Artificial Sequence
  Synthetic construct G3
gccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 11 - DNA, 4746 bp, Artificial Sequence
  Synthetic construct G4
gccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagtactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 12 - DNA, 4935 bp, Artificial Sequence
  Synthetic construct G6
gccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 13 - DNA, 4764 bp, Artificial Sequence
  Synthetic construct G4-flex
gccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttctcaaaacccaccagtcactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 14 - DNA, 4935 bp, Artificial Sequence
  Synthetic construct G6_opt
gccacccggcggtactacctgggcgccgtggaactgtcctgggactacatgcagtccgacctgggcgagctgcccgtggacgccagattccccccaagagtgcccaagtccttccccttcaacacctccgtggtgtacaagaaaaccctgttcgtggaattcaccgaccacctgttcaatatcgccaagcctcggcccccttggatgggcctgctgggccctacaatccaggccgaggtgtacgacaccgtggtgatcaccctgaagaacatggcctcccaccccgtgtccctgcacgccgtgggcgtgtcctactggaaggcctctgagggcgccgagtacgacgaccagacctcccagcgcgagaaagaggacgacaaggtgttccctggcggctcccacacctacgtgtggcaggtgctgaaagaaaacggccctatggcctccgaccctctgtgcctgacctactcctacctgtcccacgtggacctggtgaaagacctgaactccggcctgatcggcgccctgctggtgtgcagagagggctccctggccaaagaaaagacccagaccctgcacaagttcatcctgctgttcgccgtgttcgacgagggcaagtcctggcactccgagacaaagaactccctgatgcaggaccgggacgccgcctctgccagagcctggcctaagatgcacaccgtgaacggctacgtgaaccggtccctgcccggactgatcggctgccaccggaagtccgtgtactggcacgtgatcggcatgggcaccacccccgaggtgcactccatctttctggaaggccacacctttctggtgcgaaaccaccggcaggcctccctggaaatctcccctatcaccttcctgaccgctcagaccctgctgatggacctgggccagttcctgctgttttgccacatctccagccaccagcacgacggcatggaagcctacgtgaaggtggactcctgccccgaggaaccccagctgcggatgaagaacaacgaggaagccgaggactacgacgacgacctgaccgactccgagatggacgtggtgcgattcgacgacgacaactcccccagcttcatccagatcagatccgtggccaagaagcaccccaagacctgggtgcactatatcgccgccgaggaagaggactgggactacgcccctctggtgctggcccctgacgaccggtcctacaagtcccagtacctgaacaacggcccccagcggatcggccggaagtacaagaaagtgcggttcatggcctacaccgacgagacattcaagaccagagaggccatccagcacgagtccggcatcctgggccccctgctgtacggcgaagtgggcgacacactgctgatcatcttcaagaaccaggcctcccggccctacaacatctacccccacggcatcaccgacgtgcggcccctgtactctcggcggctgcctaagggcgtgaagcacctgaaggacttccccatcctgcccggcgagatcttcaagtacaagtggaccgtgaccgtggaagatggccccaccaagtccgaccccagatgcctgacccggtactactcctccttcgtgaacatggaacgggacctggccagcggcctgattggacctctgctgatctgctacaaagaatccgtggaccagcggggcaaccagatcatgtccgacaagcggaacgtgatcctgttcagcgtgttcgatgagaaccggtcctggtatctgaccgagaacatccagcggtttctgcccaaccctgccggcgtgcagctggaagatcccgagttccaggcctccaacatcatgcacagcatcaatggctacgtgttcgactccctgcagctgagcgtgtgcctgcacgaggtggcctactggtacatcctgtccatcggcgcccagaccgacttcctgtccgtgttcttctccggctacaccttcaagcacaagatggtgtacgaggataccctgaccctgttccccttctccggcgaaaccgtgttcatgtccatggaaaaccccggcctgtggatcctgggctgccacaactccgacttccggaaccggggcatgaccgccctgctgaaggtgtcctcctgcgacaagaacaccggcgactactacgaggactcttacgaggacatctccgcctacctgctgtccaagaacaacgccatcgagccccggtccttcagccagaactcccggcacccttccacccggcagaagcagttcaacgccaccaccatccccgagaacgacatcgaaaagaccgacccttggtttgcccaccggacccccatgcccaagatccagaacgtgtcctcctctgacctgctgatgctgctgcggcagtcccccacccctcacggcctgtctctgagcgacctgcaggaagctaagtacgagactttctccgacgacccctcccctggcgccatcgactccaacaactccctgtccgagatgacccacttccggccccagctgcaccactccggcgacatggtgttcacccccgagtctggcctgcagctgcggctgaacgagaagctgggcaccacagccgccaccgagctgaagaaactggacttcaaggtgtccagcacctccaacaacctgatctccaccatcccttccgacaacctggccgctggcaccgacaacacctcttccctgggccctccctccatgcccgtgcactacgactcccagctggacaccaccctgttcggcaagaagtccagccccctgaccgagtccggcggacccctgtccctgtctgaggaaaacaacgactccaagctgctggaatctggcctgatgaactcccaggaatcctcctggggcaagaatgtgtccaccctgcagagcgaccaggaagagatcgattacgacgacaccatctccgtggaaatgaagaaagaggatttcgacatctacgacgaggacgagaaccagtcccctcggtccttccagaagaaaacccggcactactttatcgccgctgtggaacggctgtgggactacggcatgtcctcctccccccacgtgctgcggaacagagcccagtccggctccgtgccccagttcaagaaagtggtgttccaggaattcacagacggcagcttcacccagcccctgtaccggggcgagctgaatgagcacctgggactgctgggaccttacatcagagctgaggtggaagataacatcatggtgacattccggaaccaggccagcagaccctactccttctactccagcctgatctcctacgaagaggaccagcggcagggcgctgagccccggaagaacttcgtgaagcccaacgagactaagacctacttctggaaggtgcagcaccacatggctcctaccaaggacgagttcgactgcaaggcctgggcctacttctccgatgtggacctggaaaaggacgtgcacagcgggctgattggcccactgctggtgtgccacaccaacaccctgaaccccgcccacggcagacaggtgacagtgcaggaattcgccctgttcttcaccatcttcgacgagacaaagtcctggtacttcaccgagaatatggaacggaactgcagagccccctgcaacatccagatggaagatcctaccttcaaagagaactaccggttccacgccatcaacggctacatcatggacaccctgcccggcctggtgatggcccaggaccagagaatccggtggtatctgctgtccatgggctccaacgagaatatccactccatccacttctccggccacgtgttcaccgtgcggaagaaagaagagtacaagatggccctgtacaacctgtaccccggcgtgttcgagacagtggaaatgctgccctccaaggccggcatctggcgggtggaatgtctgatcggcgagcatctgcacgccggaatgtctaccctgttcctggtgtactccaacaagtgccagacccccctgggcatggcctccggccacatccgggatttccagatcaccgcctctggccagtacggccagtgggcccctaagctggcccggctgcactacagcggctccatcaacgcctggtctaccaaagagcccttcagctggatcaaggtggacctgctggcccccatgatcatccatggcatcaagacccagggcgccagacagaagttctcctccctgtacatctcccagttcatcatcatgtactccctggatggcaagaagtggcagacctaccggggcaactccaccggcaccctgatggtgttcttcggcaacgtggactcctccggcatcaagcacaacatcttcaacccccccatcattgcccggtacatccggctgcaccccacccactactccatccggtctaccctgcggatggaactgatgggctgcgacctgaacagctgctccatgcctctgggcatggaatccaaggccatctccgacgcccagatcacagccagctcctacttcaccaacatgttcgccacctggtcccccagcaaggccagactgcatctgcagggccggtccaacgcttggcggcctcaggtgaacaaccccaaagaatggctgcaggtggacttccagaaaaccatgaaggtgacaggcgtgaccacccagggcgtgaaatccctgctgacctctatgtacgtgaaagagttcctgatcagctcctcccaggacggccaccagtggaccctgttctttcagaacggcaaagtgaaagtgttccagggcaaccaggactccttcacccctgtggtgaactccctggaccccccactgctgaccagatacctgcggatccacccccagtcttgggtgcaccagatcgccctgagaatggaagtgctgggatgcgaggcccaggatctgtactga

SEQ ID NO: 15 - DNA, 4746 bp, Artificial Sequence
  Synthetic construct G4_B3
gccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaatgtcaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagtactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

(19) United States
(12) Patent Application Publication
(10) Pub. No.: US 2021/0079062 A1
(43) Pub. Date: Mar. 18, 2021

(54) IN-VIVO RELEASE SUSTAINED RECOMBINANT COAGULATION FACTOR VIII AND PREPARATION METHOD THEREFOR

(71) Applicant: MOGAM INSTITUTE FOR BIOMEDICAL RESEARCH, Yongin-si, Gyeonggi-do (KR)

(72) Inventors: Kwan Yub KANG, Yongin-si (KR); Taeyoon KIM, Yongin-si (KR); Jae Hwan RYU, Yongin-si (KR); Yeon Jung KIM, Yongin-si (KR); Yong Jae KIM, Yongin-si (KR)

(73) Assignee: MOGAM INSTITUTE FOR BIOMEDICAL RESEARCH, Yongin-si, Gyeonggi-do (KR)

(21) Appl. No.: 16/961,378

(22) PCT Filed: Jan. 11, 2019
(86) PCT No.: PCT/KR2019/000479

§ 371 Date: Jul. 10, 2020

(30) Foreign Application Priority Data
Jan. 12, 2018 (KR) 10-2018-0004589

(51) Int. Cl. C07K 14/755; C12N 15/63

(52) U.S. Cl. CPC C07K 14/755; C12N 15/63

(57) ABSTRACT

An in-vivo release-sustained recombinant coagulation factor VIII and a method for making it are disclosed. The recombinant coagulation factor VIII has a biocompatible polymer that is conjugated to at least one sugar chain terminus. The in-vivo release-sustained recombinant coagulation factor VIII has a protein surface modified through a method more elaborate and safer than conventional glycoconjugation techniques and enjoys the advantage of decreasing in immunogenicity and increasing in in-vivo sustainability.

### TECHNICAL FIELD

The present invention provides an in vivo long-acting recombinant coagulation factor VIII having a structure in which a biocompatible polymer is connected to the terminus of at least one sugar chain, and a preparation method therefor.

### BACKGROUND ART

A glycoprotein refers to a protein that contains a sugar chain on the surface, the sugar chain having been connected to a protein residue in the Golgi apparatus during the protein production process. In glycoproteins, the function of a sugar chain has a significant effect on protein properties. For example, a sugar chain may improve water solubility of proteins, protect proteins from proteases, decrease immunogenicity of proteins, determine directionality of intracellular transport and localization of proteins, or affect intercellular interactions (Function and 3D Structure of the N-Glycans on Glycoproteins, Int. J. Mol. Sci. 2012, 13, 8398-8429).

Due to this importance of sugar chain, attempts have been made to improve protein properties through glycoengineering. For example, for erythropoietin (EPO), introduction of two additional N-sugar chains thereinto can improve its half-life in the body (Glycoengineering: the effect of glycosylation on the properties of therapeutic proteins, J Pharm Sci. 2005 94(8): 1626-35). In addition, it has been reported that in case of cancer cell-targeted antibody therapeutics, antibodies lacking fucose, which have been produced through glycoengineering, better induce the host's immune response and have a better therapeutic effect (Production of therapeutic antibodies with controlled fucosylation, MAbs. 2009 1(3): 230-236). These methods using glycoengineering have advantages in that generation of by-products caused by side reactions can be minimized and such methods are efficient, as compared with conventional chemical methods.

Meanwhile, regarding methods for improving glycoprotein properties through changes in sugar chain, attempts have been made to apply metabolic glycoengineering so that changes in sugar chain are implemented in a more sophisticated way; however, specific applications and commercial uses thereof have not yet been sufficient.

### DISCLOSURE OF INVENTION

### Technical Problem

An object of the present invention is to provide an in vivo long-acting recombinant glycoprotein in which a biocompatible polymer is connected to a sialic acid derivative introduced to the terminus of at least one sugar chain, preferably a coagulation factor VIII in which a biocompatible polymer is connected to a sialic acid derivative introduced to the terminus of at least one sugar chain.

Another object of the present invention is to provide a method for preparing an in vivo long-acting recombinant coagulation factor VIII, in which a metabolic labeling technique is applied so that sialic acid at the terminus of at least one sugar chain of a coagulation factor VIII is modified to a sialic acid derivative to which an azide or alkyne group is introduced.

Yet another object of the present invention is to provide a method for preparing a modified in vivo long-acting recombinant coagulation factor VIII, in which the modification is done by introducing a biocompatible polymer to the sialic acid derivative at the terminus of sugar chain.

### Solution to Problem

In order to achieve the above-mentioned object, in an embodiment of the present invention, there is provided an in vivo long-acting recombinant coagulation factor VIII, comprising a linker which is linked to a sialic acid derivative at the terminus of at least one sugar chain of a coagulation factor VIII, wherein the linker is represented by Formula 1:

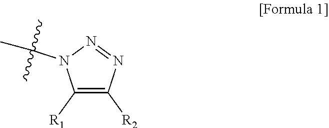

where R₁ and R₂ are each independently C₁₋₈ alkyl, C₆₋₁₂ aryl, C₃₋₆ cycloalkyl, 3- to 10-membered heterocycloalkyl, amino, (amino)C₁₋₆ alkyl, (amino)C₃₋₆ cycloalkyl, (amino)C₆₋₁₂ aryl, (amino)(3- to 10-membered heterocycloalkyl), or 5- to 12-membered heteroaryl (in which the heterocycloalkyl and the heteroaryl may each independently contain at least one heteroatom selected from the group consisting of N, S and O, and may each independently be substituted with at least one substituent selected from the group consisting of halogen, C₁₋₄ alkyl, and CF₃; and the amino may be substituted at least one substituent selected from the group consisting of H, halogen, CN, C₁₋₆ alkyl, haloC₁₋₃ alkyl or C₁₋₆ alkoxycarbonyl), or R₁ and R₂ may be combined with each other to form 8- to 16-membered heterofused ring (in which the heterofused ring may each independently contain at least one heteroatom selected from the group consisting of N, S and O, and may each independently be substituted with at least one substituent selected from the group consisting of halogen, C₁₋₄ alkyl, and CF₃); and

the linker contains a biocompatible polymer connected to at least one of R₁, R₂, or a ring formed when R₁ and R₂ are combined with each other.

In addition, in another embodiment of the present invention, there is provided a method for preparing an in vivo long-acting recombinant coagulation factor VIII, comprising the steps of: (1) introducing, into a host cell, an expression vector that contains nucleotides encoding a coagulation factor VIII, and (2) culturing the host cell in a culture medium supplemented with a derivative of sialic acid metabolite having a first functional group attached thereto, to obtain a coagulation factor VIII to which the sialic acid derivative having the first functional group attached thereto is introduced at the terminus of at least one sugar chain.

In addition, in yet another embodiment of the present invention, there is provided the method for preparing an in vivo long-acting recombinant coagulation factor VIII, further comprising the steps of: (3) removing the derivative of the sialic acid metabolite from the culture medium, after carrying out step (2); (4) adding, to the culture medium, a compound having a second functional group and a biocompatible polymer attached thereto, to allow a click reaction; and (5) collecting a coagulation factor VIII to which the biocompatible polymer is attached at the terminus of at least one sugar chain.

### Advantageous Effects of Invention

The in vivo long-acting recombinant coagulation factor VIII according to the present invention has a biocompatible polymer, such as polyethylene glycol, conjugated to its sugar chain present on the protein surface, so that immunogenicity of the coagulation factor VIII itself, which is a glycoprotein, can be decreased in the body, and in vivo long-acting performance thereof can be improved. In addition, the method for preparing an in vivo long-acting recombinant coagulation factor VIII according to the present invention utilizes a normal sugar metabolism process of a host cell of interest in glycoprotein expression using a recombinant DNA technique, thereby having advantages of high efficiency and accuracy in conjugation between the sugar chain and the biocompatible polymer. Therefore, it is possible to provide a modified recombinant coagulation factor VIII in a stable and process-efficient manner, as compared with conventional methods of converting a glycoprotein sugar chain using a sugar conjugation technique.

### BRIEF DESCRIPTION OF DRAWINGS

FIG. 1 illustrates a map of an expression vector used when transfecting scFVIII G4 B3 into a host cell in Example 1.1.

FIG. 2 illustrates results obtained by analyzing, with SDS PAGE, a purification process of scFVIII G4_B3 in Example 1.2. (lane 1: molecular weight markers, lane 2: cell culture medium concentrate, lane 3: VIII select eluate, lane 4: purified scFVIII G4 B3).

FIG. 3 illustrates a cell culture process in Example 2.1 and details of each cycle thereof.

FIGS. 4A and 4B illustrate growth curves and expression levels of scFVIII G4 B3, respectively, for a cell line expressing scFVIII G4 B3 with Ac4ManNA1 (N-(4-pentynoyl)-mannosamine-tetraacylated), and a cell line expressing scFVIII G4 B3 without Ac4ManNA1.

FIG. 5 illustrates expression levels of scFVIII G4 B3 for a cell line expressing scFVIII G4 B3 with Ac4ManNAz, and a cell line expressing scFVIII G4 B3 without Ac4ManNAz (N-azidoacetylmannosamine-tetraacylated).

FIG. 6A illustrates results obtained by analyzing, with SDS PAGE, glycoPEGylation reaction products of scFVIII G4 B3 Az and purified scFVIII G4 B3 Az (lane 1: molecular weight markers, lane 2: reaction product of scFVIII G4 B3 Az with 20 kDa DBCO PEG, lane 3: reaction product of scFVIII G4 B3 Az with 30 kDa DBCO PEG, lane 4: reaction product of scFVIII G4 B3 Az with 40 kDa DBCO PEG, lane 5: purified scFVIII G4 B3 Az).

FIG. 6B illustrates results obtained by analyzing, with SDS PAGE, a glycoPEGylation reaction product of scFVIII G4 B3 Az and purified scFVIII G4 B3 Az (lane 1: molecular weight markers, lane 2: purified scFVIII G4 B3 Az, lane 3: reaction product of scFVIII G4 B3 Az with 10 kDa DBCO PEG).

FIG. 6C illustrates results obtained by analyzing, with SDS PAGE, purified versions of scFVIII G4 B3 Az and a glycoPEGylation reaction product of scFVIII G4 B3 Az (lane 1: molecular weight markers, lane 2: purified scFVIII G4 B3 Az, lane 3: purified 20 kDa DBCO PEG-conjugated scFVIII G4 B3 Az, lane 4: molecular weight markers).

FIG. 6D illustrates results obtained by analyzing, with SDS PAGE, a purified version of a glycoPEGylation reaction product of scFVIII G4 B3 Az (lane 1: molecular weight markers, lane 2: purified 10 kDa DBCO PEG-conjugated scFVIII G4 B3 Az).

FIG. 7 illustrates results obtained by analyzing, with SDS PAGE, a product obtained in a case where a click reaction is performed by adding azide-PEG40 kDa to scFVIII G4 B3 A1 in the presence of Cu²⁺.

FIG. 8 illustrates a graph showing activity of Factor VIII remaining in the blood over time after intravenous administration of glycoPEGylated FVIII, compared to non-pegylated FVIII.

FIG. 9 illustrates a graph showing activity of Factor VIII remaining in the blood over time after subcutaneous administration of glycoPEGylated FVIII, compared to non-pegylated FVIII.

FIG. 10A illustrates a graph showing activity of Factor VIII remaining in the blood over time after subcutaneous administration of glycoPEGylated FVIII, compared to non-pegylated FVIII.

FIG. 10B illustrates a graph showing activity of Factor VIII remaining in the blood over time after subcutaneous administration of glycoPEGylated FVIII, compared to non-pegylated FVIII.

### BEST MODE FOR CARRYING OUT THE INVENTION

In an aspect of the present invention, there may be provided an in vivo long-acting recombinant coagulation factor VIII (Factor VIII, FVIII), comprising a linker which is linked to a sialic acid derivative at the terminus of at least one sugar chain of a coagulation factor VIII, wherein the linker is represented by Formula 1:

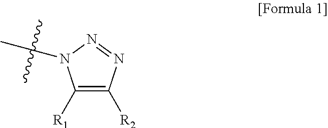

where R₁ and R₂ are each independently C₁₋₈ alkyl, C₆₋₁₂ aryl, C₃₋₆ cycloalkyl, 3- to 10-membered heterocycloalkyl, amino, (amino)C₁₋₆ alkyl, (amino)C₃₋₆ cycloalkyl, (amino)C₆₋₁₂ aryl, (amino)(3- to 10-membered heterocycloalkyl), or 5- to 12-membered heteroaryl (in which the heterocycloalkyl and the heteroaryl may each independently contain at least one heteroatom selected from the group consisting of N, S and O, and may each independently be substituted with at least one substituent selected from the group consisting of halogen, C₁₋₄ alkyl, and CF₃; and the amino may be substituted at least one substituent selected from the group consisting of H, halogen, CN, C₁₋₆ alkyl, haloC₁₋₃ alkyl or C₁₋₆ alkoxycarbonyl), or R₁ and R₂ may be combined with each other to form 8- to 16-membered heterofused ring (in which the heterofused ring may each independently contain at least one heteroatom selected from the group consisting of N, S and O, and may each independently be substituted with at least one substituent selected from the group consisting of halogen, C₁₋₄ alkyl, and CF₃); and

the linker contains a biocompatible polymer connected to at least one of R₁, R₂, or a ring formed when R₁ and R₂ are combined with each other.

The coagulation factor VIII is a glycoprotein in which a sugar chain is linked via a covalent bond to a protein residue. It is known that modifying a sugar chain on the protein surface in glycoproteins may affect protein properties, such as improving water solubility of proteins, protecting proteins from proteases, and determining directionality of proteins in intercellular interactions.

Accordingly, the present inventors used metabolic glycoengineering to conjugate a protein sugar chain to a biocompatible polymer via the linker represented by Formula 1 so that in vivo long-acting performance and stability of the coagulation factor VIII are improved, and thus have completed the present invention.

Specifically, in the present invention, the coagulation factor VIII is labeled with a biocompatible polymer using an azide-alkyne cyclization reaction (click reaction) that does not require a catalyst such as copper having cytotoxicity. Unlike other chemical reactions, the chemical reaction is feasible on a precursor even in the body because a reactive group that has been previously introduced into the precursor is azide or alkyne which allows a chemical reaction to occur without any disturbance caused by substances existing in the body.

The biocompatible polymer may be at least one selected from the group consisting of polyethylene glycol (PEG), polysialic acid (PSA), polyoxazoline, and heparosan, with polyethylene glycol being preferred.

In addition, the polyethylene glycol may have a weight average molecular weight of 5 to 40 kDa, 5 to 30 kDa, or 5 to 20 kDa. Multiple conjugation of small-sized polyethylene glycol having a molecular weight of 40 kDa or lower may decrease possibility of PEG toxicity that occurs upon conjugation of polyethylene glycol having a higher molecular weight, further improve the glycoprotein's half-life, and improve bioavailability thereof upon subcutaneous administration.

In addition, the structure of Formula 1 may be at least one selected from the group consisting of Formulas 2 to 18 and isomers thereof.

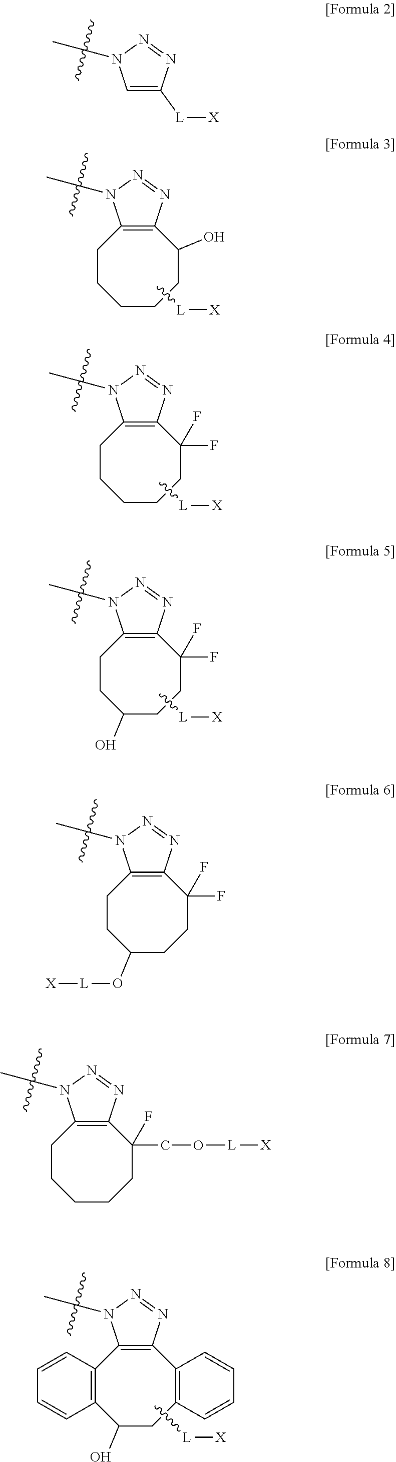

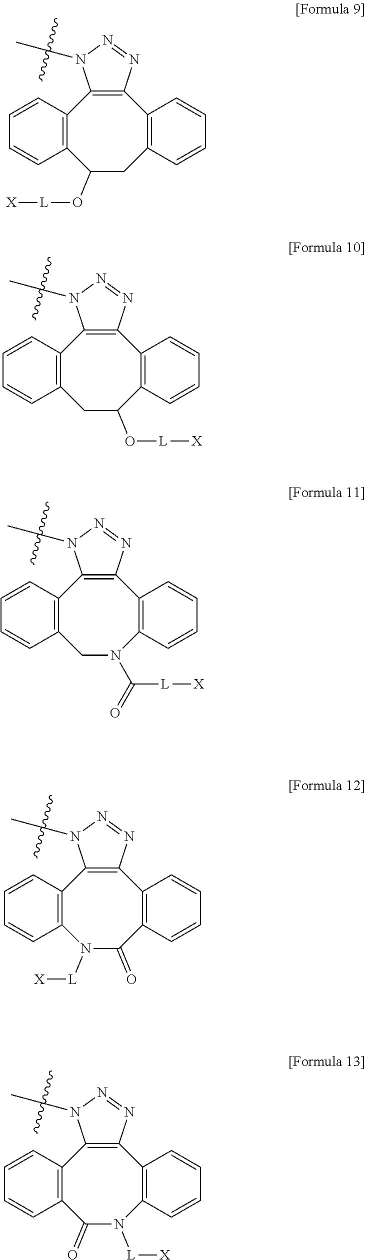

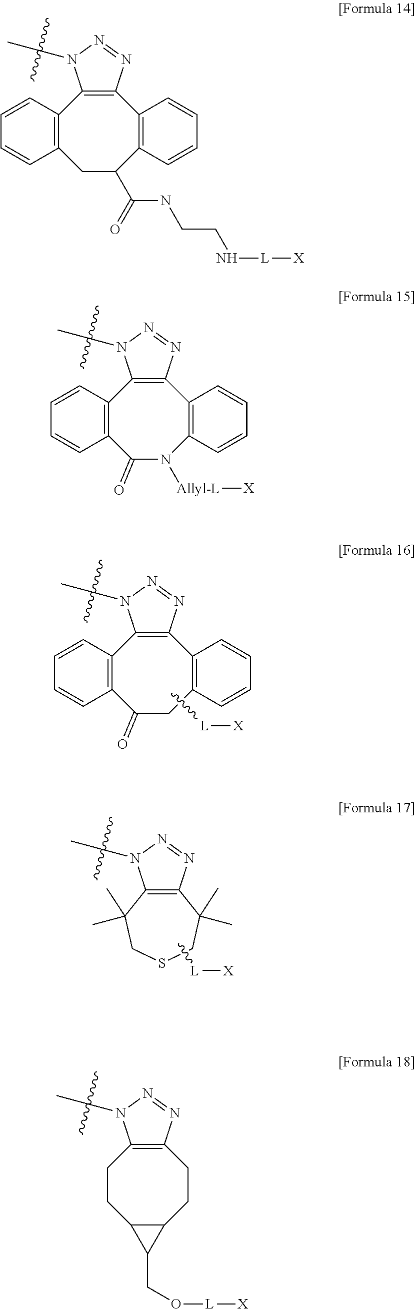

where L may be a single bond, halogen, C₁₋₈ alkyl, C₆₋₁₂ aryl, —CO—, —R₃CONR₃—, —OCONR₃—, or —R₃NCOR₃— (in which R₃ may each independently be selected from H, C₁₋₄ alkyl and C₆₋₁₂ aryl), and X is a biocompatible polymer.

Specifically, the compound may be any one of the compounds represented by Formulas 19 to 26, in which n is an integer from 100 to 3,000.

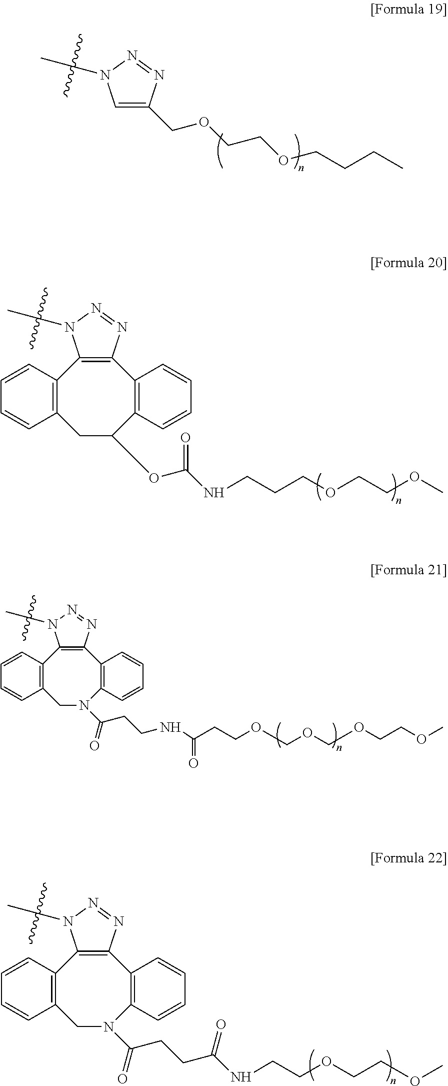

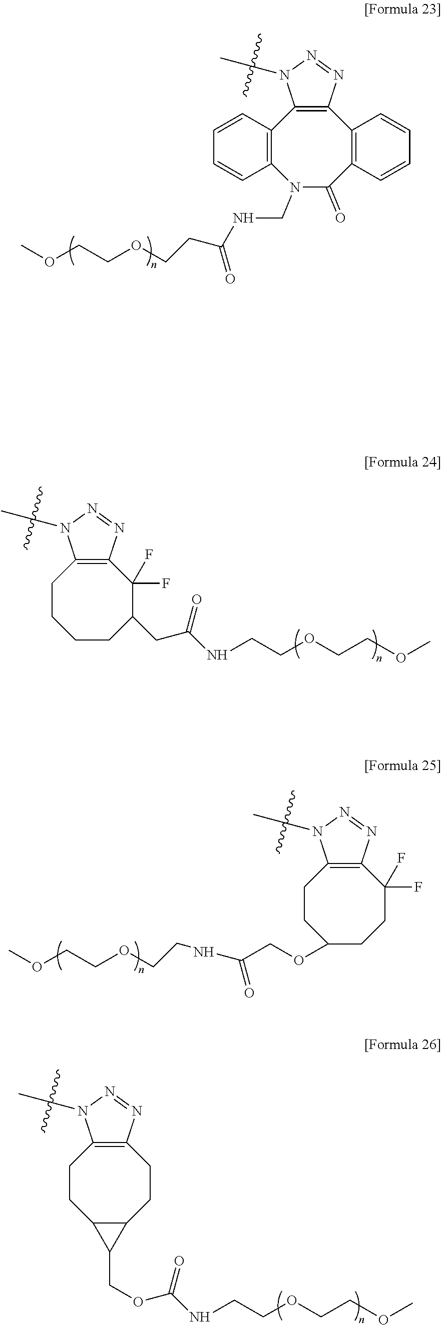

A wild-type Factor VIII protein, which is composed of A1-a1-A2-a2-B-a3-A3-C1-C2 domains, is expressed as a single-chain protein, and then matured by purine to form a heterodimer of 280 kDa containing heavy and light chains in hepatocytes. Factor VIII protein, which may be modified in the present invention, is a normal Factor VIII that may be represented by SEQ ID NO: 1, and is preferably a single-chain form of Factor VIII (scFVIII).

In addition, the Factor VIII protein contains B-domain region that is partially deleted in a contiguous or non-contiguous manner and a3-domain region that is partially deleted; and a single-chain form of Factor VIII which contains the B-domain region that is partially deleted in a contiguous or non-contiguous manner and the a3-domain region that is partially deleted may include any one of SEQ ID NO: 2 (a form obtained from SEQ ID NO: 1 by deletion of amino acid residues 789-1653 of the B domain and a3 domain regions), SEQ ID NO: 3 (a form obtained from SEQ ID NO: 1 by deletion of amino acid residues 833-1653 of the B domain and a3 domain regions), SEQ ID NO: 4 (a form obtained from SEQ ID NO: 1 by deletion of amino acid residues 903-1653 of the B domain and a3 domain regions), SEQ ID NO: 5 (a form obtained from SEQ ID NO: 1 by deletion of amino acid residues 966-1653 of the B domain and a3 domain regions), SEQ ID NO: 6 (a form obtained from SEQ ID NO: 1 by deletion of amino acid residues 903-1653 and 1643-1653 of the B domain and a3 domain regions), SEQ ID NO: 7 (a form obtained from SEQ ID NO: 1 by deletion of amino acid residues 966-1653 of the B domain and a3 domain regions), and SEQ ID NO: 8 (a form obtained from SEQ ID NO: 4 by isoleucine-to-cysteine substitution at amino acid residue 782).

In addition, Factor VIII protein may include variously modified peptides, that is, variants. The modification may be carried out by substitution, deletion, or addition of one or more amino acids within a range that does not alter the function of Factor VIII. These various peptides may be 90%, 91%, 92%, 93%, 94%, 95%, 96%, 97%, 98%, or 99% or more identical to any one of the amino acid sequences of SEQ ID NOs: 2 to 8.

In addition, a sugar chain in the Factor VIII protein, to which the linker is connected, may be included at at least one position of amino acid residues 42, 239, 582, 757, 784, 828, 900, 963, 1001, 1005, 1055, 1066, 1185, 1255, 1259, 1282, 1300, 1412, 1442, 1810, and 2118, based on the amino acid sequence of SEQ ID NO: 1. As such, Factor VIII proteins including single-chain Factor VIII (scFVIII) have many sugar chains on the surface, and thus are suitable for modifying the protein surface by applying metabolic glycoengineering according to the present invention.

In addition, in another aspect of the present invention, there may be provided a method for preparing an in vivo long-acting recombinant coagulation factor VIII, comprising the steps of: (1) introducing, into a host cell, an expression vector that contains nucleotides encoding a coagulation factor VIII, and (2) culturing the host cell in a culture medium supplemented with a derivative of a sialic acid metabolite having a first functional group attached thereto.

In step (1), the sequence of the coagulation factor VIII may be any one of the amino acid sequences of SEQ ID NOs: 2 to 8, and the nucleic acid sequence of the nucleotides may be any one of the nucleotide sequences of SEQ ID NOs: 9 to 15. Further, the nucleotides may additionally contain a signal sequence or a leader sequence.

As used herein, the term “signal sequence” refers to a nucleic acid encoding a signal peptide that directs secretion of a fusion protein. The signal peptide is translated in a host cell and then cleaved. Specifically, the signal sequence of the present invention is a polynucleotide encoding an amino acid sequence that initiates movement of a protein through the endoplasmic reticulum (ER) membrane. Signal sequences useful in the present invention include antibody light chain signal sequences, for example, antibody 14.18 (Gillies et al., J. Immunol. Meth 1989. 125: 191-202), antibody heavy chain signal sequences, for example, MOPC141 antibody heavy chain signal sequence (Sakano et al., Nature, 1980. 286: 676-683), and other signal sequences known in the art (for example, Watson et al., Nucleic Acid Research, 1984. 12: 5145-5164).

Characteristics of signal peptides are well known in the art. The signal peptides typically contain 16 to 30 amino acid residues and may contain greater or fewer amino acid residues. Typical signal peptides consist of three regions, that is, a basic N-terminal region, a central hydrophobic region, and a more polar C-terminal region.

The central hydrophobic region contains 4 to 12 hydrophobic residues that immobilize a signal sequence across a membranous lipid bilayer during movement of an immature polypeptide. After the initiation, the signal sequence is cleaved within the lumen of ER by cellular enzymes, commonly known as signal peptidases. Here, the signal sequence may be a signal sequence of tissue plasminogen activator (tPa), a signal sequence of Herpes simplex virus glycoprotein D (HSV gD), or a secretion signal sequence of growth hormone. Preferably, a secretion signal sequence used in higher eukaryotic cells including mammals and the like may be used. In addition, for the secretion signal sequence of the present invention, a signal sequence included in wild-type IL-7 may be used or may be substituted with a codon having a high expression frequency in a host cell and used.

In addition, the expression vector is a vector that can be introduced into a host cell and can be recombined with and inserted into the host cell's genome. The vectors include linear nucleic acids, plasmids, phagemids, cosmids, RNA vectors, viral vectors, and analogs thereof. Examples of the viral vectors include, but are not limited to, retroviruses, adenoviruses, and adeno-associated viruses. In addition, the plasmids may contain a selection marker such as an antibiotic resistance gene, and plasmid-containing host cells may be cultured under selective conditions.

In addition, as used herein, the term “host cell” refers to a prokaryotic or eukaryotic cell into which a recombinant expression vector can be introduced. As used herein, the terms “transduced”, “transformed”, and “transfected” refer to introduction of a nucleic acid (for example, a vector) into cells using techniques known in the art. Appropriate host cells may be used for expression and/or secretion of Factor VIII proteins by being transduced or transfected with the nucleotide sequences of the present invention. Preferred host cells that can be used in the present invention include Escherichia coli, immortal hybridoma cells, NS/0 myeloma cells, 293 cells, Chinese hamster ovary cells (CHO cells), HeLa cells, human amniotic fluid derived cells (CapT cells), or COS cells. For example, the most preferred host strain used to express a Factor VIII protein in the present invention is CHO-S, and the genes and methods of using the same are known in the art.

In addition, in step (2), supplementation with the derivative of a sialic acid metabolite having a first functional group attached thereto when culturing the host cell is made so as to cause the sialic acid derivative having the first functional group attached thereto to be introduced to the terminus of a sugar chain of the coagulation factor VIII via the sialic acid metabolic pathway of the host cell protein. That is, in step (2), the coagulation factor VIII can be modified with the sialic acid derivative having the first functional group attached thereto.

Here, the first functional group may be an azide group or alkyne group. In addition, the derivative of sialic acid metabolite may contain an acetyl group, or an alkyl group such as an ethyl group and a propyl group in order to be efficiently delivered into cells. The alkyl group forms an ester bond with a hydroxyl group of the sialic acid metabolite having a first functional group attached thereto, to facilitate cell permeation thereof; and after the cell permeation, the alkyl group is removed by intracellular esterases. Thus, the alkyl group does not prevent the supplemented sialic acid metabolite from replacing part of the sugar chain.

In addition, in step (2), the derivative of a sialic acid metabolite, which can replace the sialic acid metabolite (an intermediate substance in the sialic acid biosynthetic pathway of the coagulation factor VIII) may be at least one compound selected from the group consisting of Formulas 27 to 32 and isomers thereof:

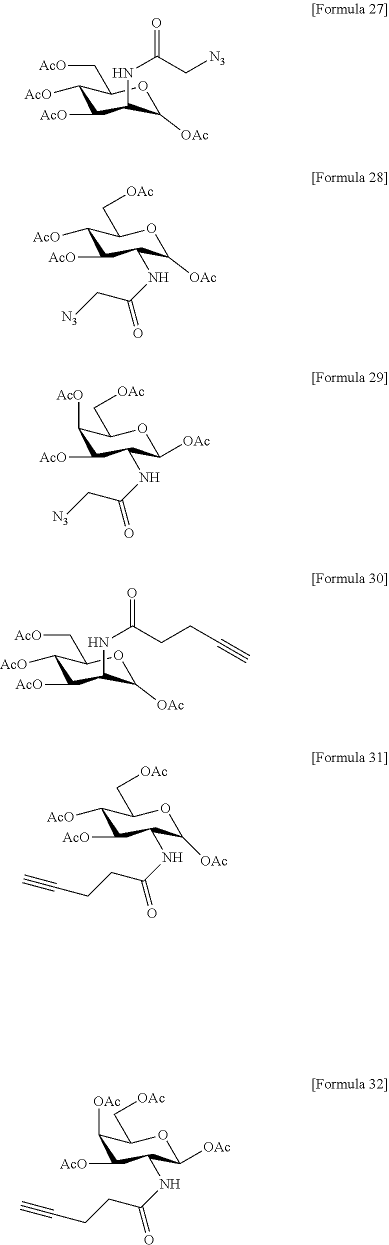

Meanwhile, in step (2), the derivative of the sialic acid metabolite may be supplemented in a concentration of 0.1 to 300 uM (micro mole) when the animal cell-derived host cell has a cell concentration of 1.0×10⁵ or higher. Since the method according to the present invention utilizes the cell's sugar metabolism process, the sialic acid metabolite having a first functional group attached thereto may replace not only part of the target coagulation factor VIII, but also part of metabolites resulting from metabolism associated with all glycoproteins in the host cell. Therefore, in order to introduce, to the coagulation factor VIII, the first functional group at a high rate for the purpose of sugar chain modification, it is preferred that the derivative of sialic acid metabolite is supplemented when the host cell has a high cell concentration, preferably a cell concentration of 1.0×10⁵ or higher.

It is preferable that the host cell is cultured as follows. During a growth phase of the host cell, culture thereof is performed without supplementation with the derivative of sialic acid metabolite; after a certain level of cell growth is achieved, the cell culture condition is changed to a condition under which the cell growth rate is decreased and the cell survival is maintained; and then the host cell is cultured in a culture medium supplemented with the derivative of sialic acid metabolite having a first functional group. The sialic acid metabolite, which is supplemented at a time when growth of the host cell is decreased, can be introduced to the sialic acid at the sugar chain of a target protein at a higher rate than when the cell is growing.

Here, as a condition under which the cell growth rate is decreased, there is a method of adjusting the temperature to a low temperature or limiting nutrient components of the culture medium. Specifically, when the culture temperature is changed from 37° C. during the cell growth phase to 29° C. to 35° C. at the time of supplementing the derivative of sialic acid metabolite, the cell growth may be slowed, whereas the expression of the target protein (coagulation factor VIII) may be continued for 10 days or longer. In addition, it is possible to decrease the concentration of nutrients such as biological growth factors, vitamins, and essential amino acids in the cell culture medium. In addition, in case of subjecting a sugar chain of Factor VIII to metabolic labeling, a substance such as butyric acid is added to affect the cell cycle, so that the cell growth is stopped and the expression of Factor VIII is maintained as it is, thereby increasing a rate of the sialic acid derivative having a first functional group to be introduced to the sugar chain of Factor VIII.

In addition, the method may further comprise the steps of: (3) removing the derivative of the sialic acid metabolite from the culture medium, after purifying, from step (2), the coagulation factor VIII whose sugar chain contains the sialic acid derivative having a first functional group attached thereto; (4) adding, to the culture medium, a compound having a second functional group and a biocompatible polymer attached thereto, to allow a click reaction to proceed; and (5) collecting a coagulation factor VIII to which the biocompatible polymer is attached at the terminus of at least one sugar chain.

In order to allow the first functional group of the sialic acid derivative in the coagulation factor VIII to react with the second functional group, it is necessary to remove the derivative of sialic acid metabolite which is present in excess in the culture medium.

Here, the first functional group may be an azide group or alkyne group. In addition, the second functional group is an alkyne group or a cycloalkyne group when the first functional group is an azide group; and the second functional group may be an azide group when the first functional group is an alkyne group.

Specifically, in step (4), conjugation of the biocompatible polymer may be performed using a click reaction for the sialic acid derivative having an azide group or alkyne group which has been introduced to a sugar chain of the target protein according to step (2) in the method of the present invention. For example, when the derivative of sialic acid metabolite having an azide group attached thereto is used in step (2), a compound having an alkyne group or a cycloalkyne group and a biocompatible polymer attached thereto may be used in step (4); and when the derivative of sialic acid metabolite having an alkyne group attached thereto is used in step (2), a compound having an azide group and a biocompatible polymer attached thereto may be used in step (4).

In step (4), the compound having a second functional group and a biocompatible polymer attached thereto is preferably at least one compound selected from the group consisting of Formulas 33 to 49 and isomers thereof:

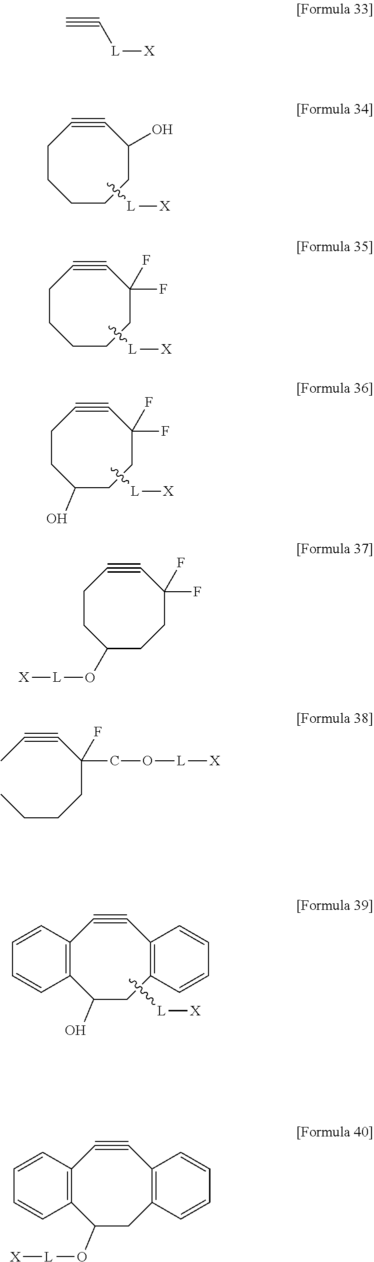

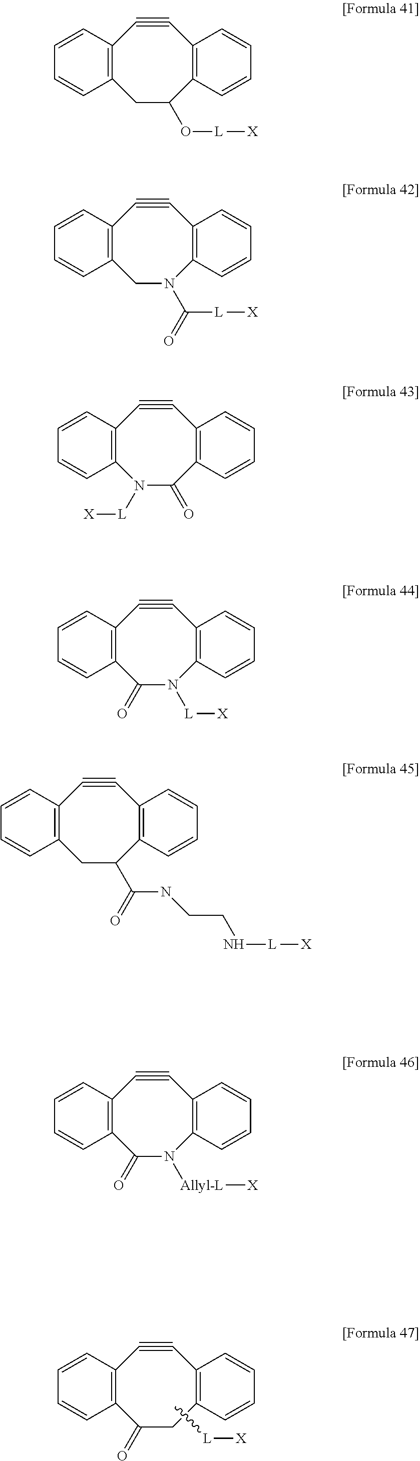

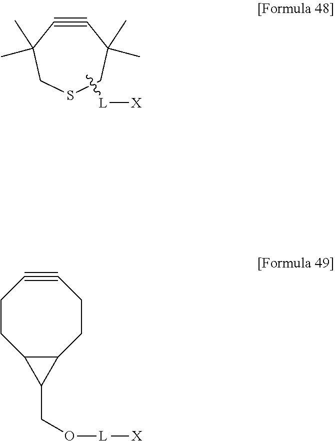

where L may be a single bond, halogen, C₁₋₈ alkyl, C₆₋₁₂ aryl, —CO—, —R₃CONR₃—, —OCONR₃—, or —R₃NCOR₃— (in which R₃ may each independently be selected from H, C₁₋₄ alkyl and C₆₋₁₂ aryl), and X is a biocompatible polymer.

In addition, the coagulation factor VIII collected in step (4) contains a linker represented by Formula 1 which is linked to the sialic acid derivative at the terminus of at least one sugar chain, and specific properties or examples thereof are as described above for the in vivo long-acting recombinant coagulation factor VIII according to the present invention:

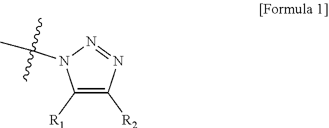

where R₁ and R₂ are each independently C₁₋₈ alkyl, C₆₋₁₂ aryl, C₃₋₆ cycloalkyl, 3- to 10-membered heterocycloalkyl, amino, (amino)C₁₋₆ alkyl, (amino)C₃₋₆ cycloalkyl, (amino)C₆₋₁₂ aryl, (amino)(3- to 10-membered heterocycloalkyl), or 5- to 12-membered heteroaryl (in which the heterocycloalkyl and the heteroaryl may each independently contain at least one heteroatom selected from the group consisting of N, S and O, and may each independently be substituted with at least one substituent selected from the group consisting of halogen, C₁₋₄ alkyl, and CF₃;

and the amino may be substituted at least one substituent selected from the group consisting of H, halogen, CN, C₁₋₆ alkyl, haloC₁₋₃ alkyl or C₁₋₆ alkoxycarbonyl), or R₁ and R₂ may be combined with each other to form 8- to 16-membered heterofused ring (in which the heterofused ring may each independently contain at least one heteroatom selected from the group consisting of N, S and O, and may each independently be substituted with at least one substituent selected from the group consisting of halogen, C₁₋₄ alkyl, and CF₃); and the linker contains a biocompatible polymer connected to at least one of R₁, R₂, or a ring formed when R₁ and R₂ are combined with each other.

### MODE FOR THE INVENTION

Hereinafter, the present invention will be described in more detail by way of examples. However, the following examples are only for illustrating the present invention, and the scope of the present invention is not limited thereto. Specifically, the technology of the present invention will be described for scFVIII G4 B3 that is one scFVIII of various scFVIII's produced in a patent application (PCT/KR2017/006633) filed by the present inventor(s) prior to this patent application. However, the reason why one scFVIII is selected in describing the technology of the present invention is not because the selected scFVIII has particular advantages, but because the technical description to be presented is made clearer merely as an embodiment.

### Example 1. Preparation of Recombinant Single-Chain (Sc) Factor VIII

### Example 1.1. Production of scFVIII-Expressing Cell Line

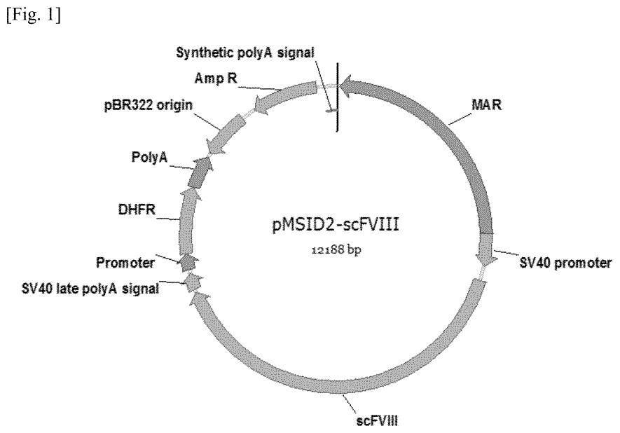

A vector (see FIG. 1), which expresses a DNA nucleotide sequence (the nucleotide sequence of SEQ ID NO: 15) encoding scFVIII G4 B3 (the amino acid sequence of SEQ ID NO: 8) described in a patent application (PCT/KR2017/006633) filed by the present inventors, was used to transform a CHO-S cell line. After the transformation, the CHO-S cell line was passaged in the presence of methotrexate (MTX) of 500 mM or higher, and a scFVIII G4 B3-high-expressing cell line (CHO-S G4 B3) was selected. The selected cell line, CHO-S G4 B3 clone, was propagated in CD FortCHO cell culture medium, and then stored in a nitrogen tank.

### Example 1.2. Culture and Purification of scFVIII

The selected scFVIII G4 B3-high-expressing cell line, CHO-S G4 B3, was added to CD FortCHO medium so that the number of cells was 3×10⁵/mL, and then cell culture was performed for 4 days. After the cell culture, the culture medium was collected and the expressed scFVIII G4 B3 was purified. In order to purify scFVIII from the culture medium, 5 M NaCl solution was added to the culture medium, in which scFVIII G4 B3 was expressed, so that NaCl has a final concentration of 1.0M, and then the culture medium was passed through a 0.2 μm membrane filter to remove fine suspended matters. Then, the filtrate was injected into the FVIII select resin (GE Healthcare) equilibrated with FVIII select equilibrium solution (20 mM histidine, 5 mM CaCl₂, 1 M NaCl, 0.02% Tween®80, pH 7.0) so that scFVIII G4 B3 was attached thereto. Then, VIII select equilibration buffer was used to remove impurities, and VIII select elution buffer (20 mM histidine, 5 mM CaCl₂, 0.9 M arginine, 45% propylene glycol, 0.02% Tween®80, pH 6.5) was used to perform elution (using affinity chromatography). Next, the VIII select eluate was diluted 5 times or higher with an equilibration buffer solution for ion exchange chromatography (20 mM histidine, 5 mM CaCl₂, 0.02% Tween®80, pH 7.0), and then injected into SP Sepharose fast flow ion exchange chromatography resin (GE Healthcare) equilibrated with the equilibrium solution for ion exchange chromatography.

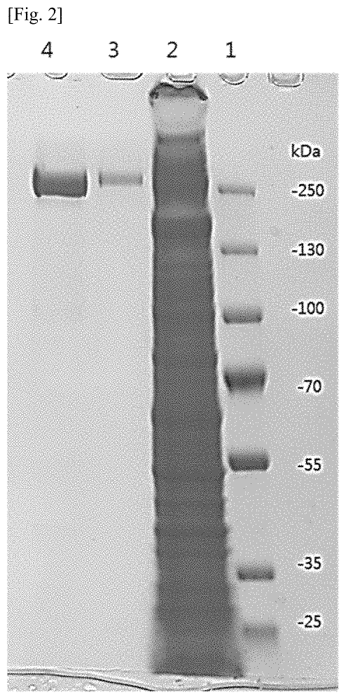

Thereafter, the equilibrium solution for ion exchange chromatography was used to remove impurities, and an ion exchange chromatography eluate (20 mM histidine, 5 mM CaCl₂, 0.02% Tween® 80, 400 mM NaCl, pH 6.5) was used to elute pure scFVIII G4 B3. FVIII activity of the purified scFVIII G4 B3 was quantified by the CS assay described in Example 1.3. below. The results of SDS PAGE performed to analyze intermediate substances at respective purification stages are as illustrated in FIG. 2.

### Example 1.3. Measurement of scFVIII Activity

The purified scFVIII G4_B3 was measured for FVIII activity by CS method.

To measure activity by the chromogenic method, a chromogenic assay kit sold by CHROMOGENIX was used with an endpoint method. The test was conducted using the test method provided by the manufacturer which was modified as follows so as to be suitable for this test. Specifically, samples were diluted to fall within a standard range (0.25 to 1 IU/mL) using 1× dilution buffer. Standards (calibration plasma) were prepared using 1× dilution buffer to have concentrations of 0, 0.25, 0.5, 1 IU/mL. 10 mL each of the thus prepared samples and standards was diluted with 790 mL of 1× dilution buffer. Then, 50 uL each of the dilutions was dispensed into a 96-well plate, and left to stand at 37° C. for 5 minutes. Using an automatic dispenser pipette, 50 uL each of agent samples was dispensed into each well and reaction was allowed to occur at 37° C. for 2 minutes. 50 uL of substrate was dispensed into each well, and then left to stand at 37° C. for 2 minutes before development. To the developed sample was added 50 uL of 2% citric acid, to stop the reaction. Absorbance was measured at a wavelength of 405 nm to draw a linear calibration curve. Then, the sample's absorbance was put into the calibration curve to measure activity of the sample, and the results are shown in Table 1 below. The purified scFVIII G4_B3 had activity of about 7,000 IU/mg, which is similar to that of a commercially available recombinant FVIII (Advate®) intended to treat hemophilia. Thus, it was determined that the purified scFVIII G4_B3 has a level of activity sufficient to be used as a therapeutic agent for hemophilia.

TABLE 1

| | Specific |
|---|---|
| Sample | activity |
| substance | (IU/mg) |
| scFVIII G4_B3 | 7,166 |
| FVIII | 6,000 to 7,000 |

### Example 2. Labeling of scFVIII Using Glyco-Metabolic Labeling

### Example 2.1. Labeling of scFVIII with Ac4ManNA1

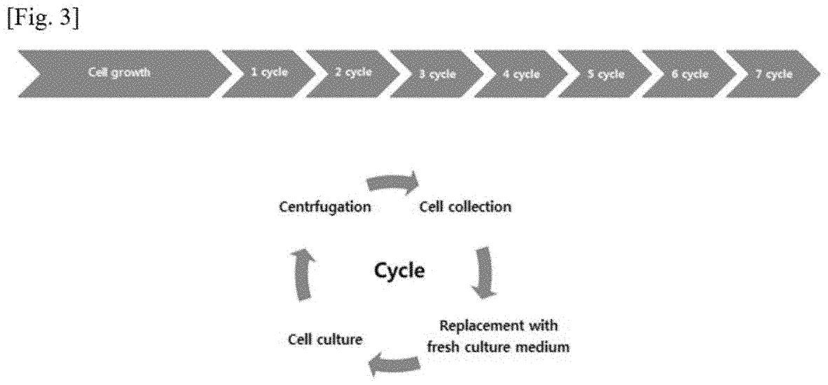

CHO-S scFVIII G4 B3 cell line was suspended in cell culture medium (CD FortiCHO) to a cell concentration of 0.3 to 3.0×10⁶, and then cell culture was performed for 4 days with stirring at 140 rpm under a condition of 37° C. and 5% CO₂. When the cell concentration reached a certain level, Ac4ManNA1 (N-(4-pentynoyl)-mannosamine-tetraacylated) was supplemented to a final concentration of 100 uM, and then cell culture was performed for 24 hours with stirring at 140 rpm under a condition of 31° C. and 5% CO₂. After completion of the cell culture, the cell culture medium was centrifuged at 1,000 rpm to remove the culture medium, and then the collected cells were suspended in cell culture medium (CD FortiCHO) supplemented with Ac4ManNA1 to a final concentration of 100 uM, so that the cell concentration was adjusted to a level of about 0.1 to 1.5×10⁷. Then, culture was performed for 24 hours with stirring at 140 rpm under a condition of 31° C. and 5% CO₂. Subsequently, a cell culture cycle was allowed to proceed three or more times (pseudo-perfusion cycle), each cycle involving a process in which culture was performed under the same condition as above while replacing, every 24 hours, the cell culture medium with fresh cell culture medium supplemented with Ac4ManNA1 to a final concentration of 100 uM. The cell culture process is illustrated in FIG. 3.

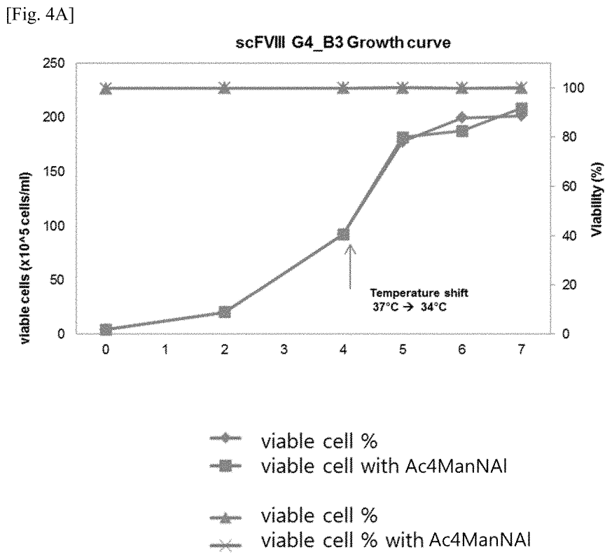

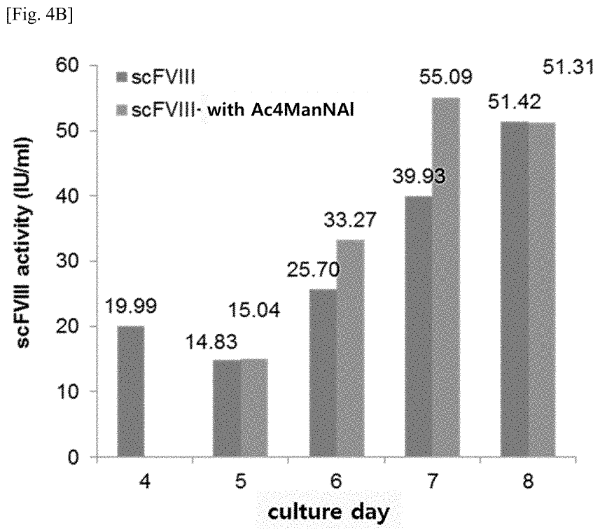

In order to determine an effect of supplementation of the medium with Ac4ManNA1 on the growth of scFVIII G4 B3 cells and the expression of scFVIII G4 B3, after the initial 4-day cell growth period, subsequent cell culture cycles were allowed to proceed with cell culture medium not supplemented with Ac4ManNA1, so that comparison on the cell growth and the expression of scFVIII G4 B3 was made in terms of FVIII activity. Supplementation with Ac4ManNA1 at 100 uM had no significant effect on the growth of CHO-S scFVIII G4 B3 cells and the expression of scFVIII G4 B3. The initial 4-day culture without Ac4ManNA1 caused the cell concentration to reach a level of about 1.0×10⁷ cells/mL, and the subsequent culture performed after supplementation with Ac4ManNA1 at 100 uM caused the cell concentration to reach a level of 1.7×10⁷ cells/mL. Thereafter, the cell concentration was maintained at a constant level. As shown in the growth curves of FIG. 4A and the expression level graphs of FIG. 4B, it was identified that scFVIII G4 B3 was expressed at a similar level regardless of the supplementation with Ac4ManNA1 at 100 uM. Accordingly, it can be seen that the supplementation with Ac4ManNA1 in a concentration range used for culture had no significant effect on the growth of the scFVIII G4 B3-expressing cell line and the expression level of scFVIII G4 B3.

### Example 2.2. Labeling of scFVIII with Ac4ManNAz

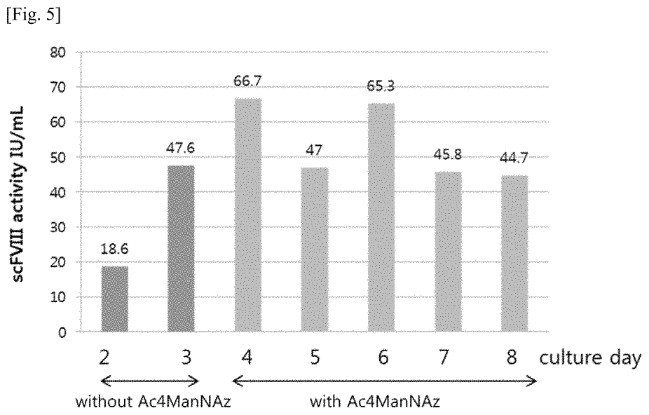

The ScFVIII G4 B3 cells were cultured in a similar manner while being supplemented with Ac4ManNAz (N-azidoacetylmannosamine-tetraacylated) at 100 uM in place of Ac4ManNA1 in the same method as in Example 2.1. After the initial 3-day culture, during each cycle of passage, culture was performed with passage culture medium whose volume corresponds to 50% of that of the passage culture medium in Example 2.1., to maintain a high cell concentration. Similar to Ac4ManNA1, the supplementation with Ac4ManNAz at 100 uM had no significant effect on the cell growth and the expression of scFVIII. Regarding the cell concentration, starting from day 4 after the culture was shifted to 31° C., the culture medium was replaced daily with the equal volume of fresh medium as in Example 2.1. The scFVIII G4 B3 cells were maintained at a level of 1.5×10⁷ or higher until the end of the culture; and the cell viability was maintained at 90% or higher until the end of the culture. As illustrated in FIG. 5, the expression level of scFVIII G4 B3 was similar to that obtained in a case where the scFVIII G4 B3 cells are cultured while being supplemented with Ac4ManNA1 in Example 2.1. In conclusion, it was found that the supplementation with Ac4ManNAz at a concentration used for culture had neither a significant effect on the cell growth of the scFVIII G4 B3 cell line nor the expression of scFVIII G4 B3.

### Example 3. Preparation of Modified scFVIII

### Example 3.1. Purification of scFVIII Labeled with Azide or Alkyne Group and Activity Measurement Thereof

scFVIII G4 B3Az (labeled with Ac4ManNAz) and scFVIII G4 B3A1 (labeled with Ac4ManNA1), which had been labeled with azide and alkyne groups, respectively, using Ac4ManNAz and Ac4ManNA1, were purified in the same method as described in Example 1.2. above for scFVIII G4 B3. Activity measurement of the purified scFVIII G4 B3Az and scFVIII G4 B3A1 was also performed in the same method as in Example 1.3. above, and the results are shown in Table 2 below. CS activity measurement shows that specific activity of the purified scFVIII G4 B3Az and scFVIII G4 B3A1 was similar to that of scFVIII G4 B3 as shown in Table 2. From these results, it was found that labeling of scFVIII G4 B3 with Ac4ManNAz or Ac4ManNA1 through glycol-metabolic labeling had no effect on activity of scFVIII G4.

TABLE 2

| | Specific |
|---|---|
| | activity |
| Sample substance | (IU/mg) |
| scVIII G4 B3 | 7,166 |
| scFVIII G4 B3Az | 6,600 |
| (scFVIII G4 B3 labeled with Ac4ManNAz) | |
| scFVIII G4 B3Al | 5,860 |
| (scFVIII G4 B3 labeled with Ac4ManNAl) | |

### Example 3.2. Preparation of glycoPEGylated scFVIII Through Click Chemistry

### Example 3.2.1. Preparation of glycoPEGylated scFVIII with Cu²⁺ Free Click Chemistry

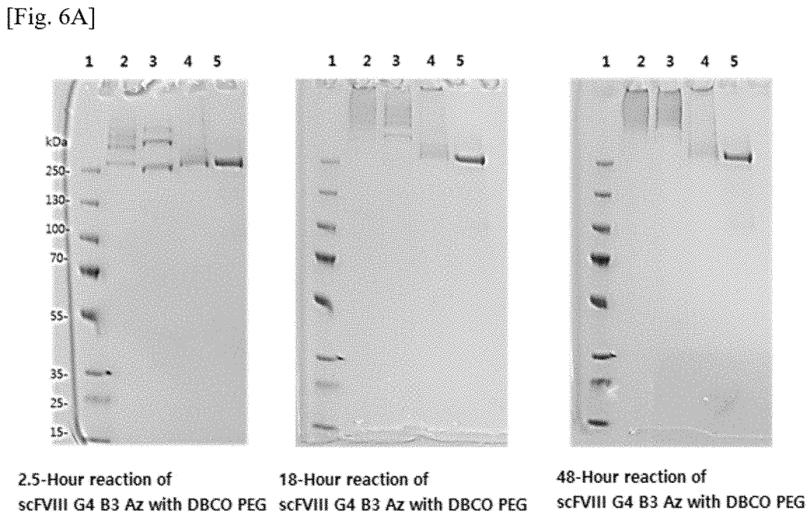

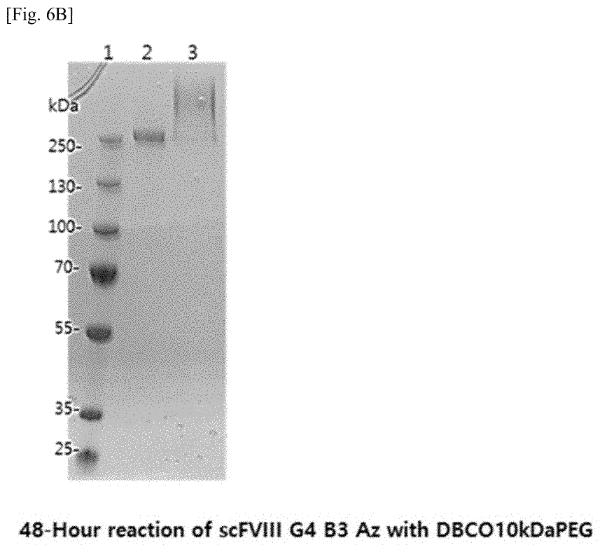

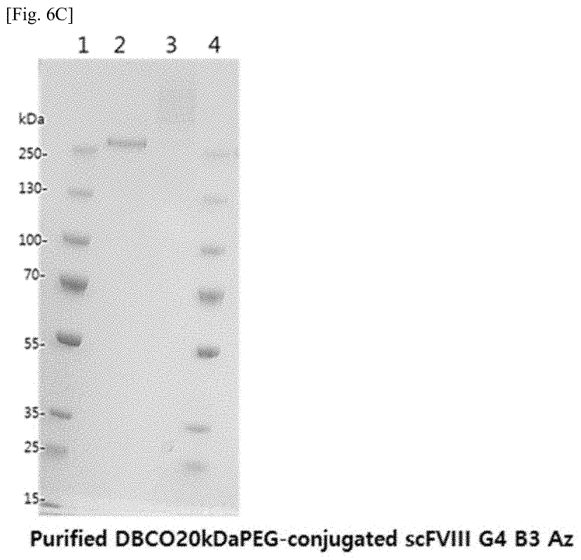

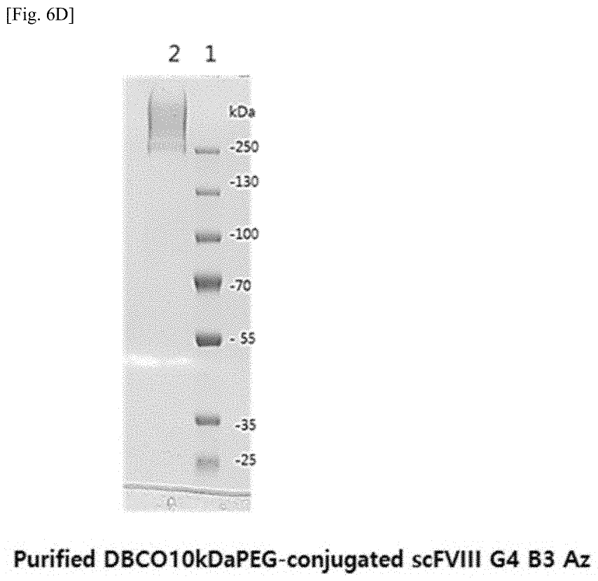

After exchange into a conjugation buffer (20 mM HEPES, 150 mM NaCl, 5 mM CaCl₂, 0.01% (w/v) Tween® 80, pH 7.5), to scFVIII G4 B3Az purified in Example 3.1. was added DBCO-PEG10 kDa, DBCO-PEG20 kDa, DBCO-PEG30 kDa so that each molar ratio of DBCO-PEG (10 kDa, 20 kDa, 30 kDa) to scFVIII G4 B3Az is 100:1. Then, reaction was allowed to proceed at 4° C. for 48 hours, and thus glycoPEGylated scFVIIIs (DBCOPEG10 kDa-scFVIII G4 B3Az, DBCOPEG20 kDa-scFVIII G4 B3Az, DBCO-PEG30 kDa-scFVIII G4 B3Az) were produced. As illustrated in FIGS. 6A and 6B, it was identified that most of the scFVIII G4 B3 Az, which is scFVIII whose sugar has been modified with Ac4ManNAz, is conjugated with DBCO PEG. In addition, it was identified that consistent with the fact that a plurality of sialic acid molecules in a sugar chain of scFVIII are modified with Ac4ManNAz, multiple conjugation occurs to form a mixture of polymer conjugates. The above DBCOPEG10 kDa-, DBCOPEG20 kDa-, and DBCOPEG30 kDa-scFVIII's were purified by the same method. Specifically, the reaction conjugates were injected into a DEAE Sepharose fast flow ion exchange resin equilibrated with an equilibration buffer (20 mM HEPES, 5 mM CaCl₂, 0.02% (w/v) Tween® 80) for attachment, and then unPEGylated scFVIII G4 B3Az was removed with a washing buffer (20 mM HEPES, 5 mM CaCl₂, 0.02% (w/v) Tween® 80). Then, glycoPEGylated scFVIII G4 B3Az was eluted with an elution buffer (20 mM HEPES, 5 mM CaCl₂, 500 mM NaCl, 0.02% (w/v) Tween® 80). The glycoPEGylation reaction products and purified glycoPEGylated scFVIII's were checked through SDS PAGE, and the results are illustrated in FIG. 6C. Activity of the eluted glycoPEGylated scFVIII's (DBCOPEG10 kDa-scFVIII, DBCOPEG20 kDa-scFVIII, DBCOPEG30 kDa-scFVIII) was measured by the CS method as described in Example 1.3. above. As shown in Table 3, their activity was not decreased as compared with scFVIII G4_B3Az before glycoPEGylation. As a result, it was found that labeling with Ac4ManNAz metabolism and subsequent glycoPEGylation do not decrease activity of scFVIII G4 B3Az.

TABLE 3

| | Specific |
|---|---|
| | activity |
| Sample substance | (IU/mg) |
| scFVIII G4_B3Az | 6,600 |
| DBCOPEG10 kDa-scFVIII G4_B3Az | 6,700 |
| DBCOPEG20 kDa-scFVIII G4_B3Az | 6,100/5,800 |
| DBCOPEG30 kDa-scFVIII G4_B3Az | 5,000 |

### Example 3.2.2. Production of glycoPEGylated Single-Chain FVIII Using Cu²⁺ Click Chemistry

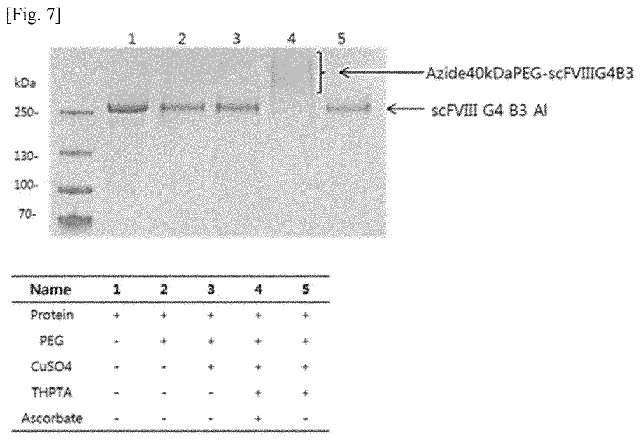

After exchange into a conjugation buffer (20 mM HEPES, 150 mM NaCl, 5 mM CaCl₂, 0.01% (w/v) Tween®80), to scFVIII G4 B3 A1 purified in Example 3.1. was added azide-PEG40 kDa (Nanocs, PG1-AZ-40k) so that a molar ratio of azide-PEG40 kDa to scFVIII G4 B3 A1 is 1:100. To initiate conjugation reaction between scFVIII G4 B3 A1 and azide-PEG40 kDa, Cu′ and THPTA were added so that Cu²⁺ had a final concentration of 0.1 mM and THPTA had a final concentration of 0.5 mM; and then ascorbate was added so that ascorbate has a final concentration of 5 mM. Then, reaction was allowed to proceed at room temperature for 24 hours. The conjugation reaction product was checked through SDS PAGE, and the results are illustrated in FIG. 7. Most of scFVIII G4 B3 A1's were conjugated. This means that most of the sugar chains of scFVIII G4 B3 were modified with alkyne by way of Ac4ManNA1. After the 24-hour conjugation reaction, activity of the conjugation reaction product was measured by the CS method as described in Example 1.3. It was identified that the conjugation product lost 50% or higher of activity as compared with the activity of FVIII in scFVIII G4 B3 A1 before the conjugation reaction. These results are presumed to be because free radicals, generated by reaction of Cu′ and ascorbate which have been added for the conjugation reaction between alkyne-labeled scFVIII B3 G4A1 and azide-PEG40 kDa, resulted in denaturation of the scFVIII G4 B3 A1 protein.

### Example 4 Evaluation of In Vivo Long-Acting Performance of scFVIII Having Polyethylene Glycol-Labeled Sugar Chain

### Example 4.1. Pharmacokinetics after Administration by Intravenous Injection

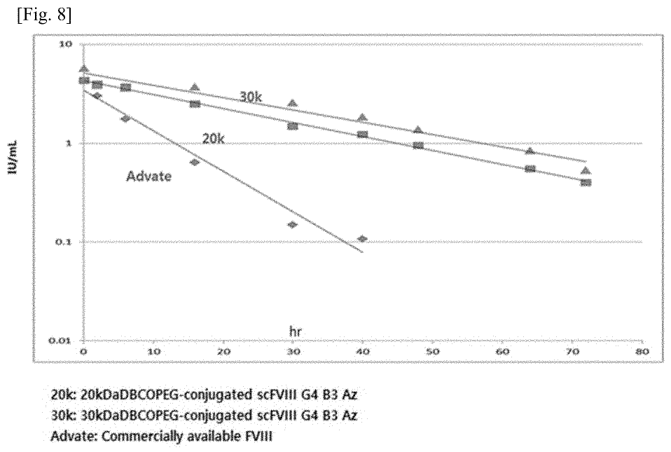

Hemophilia mice were administered by intravenous injection of scFVIIIs (glycoPEGylated scFVIII) labeled with polyethylene glycol which had been prepared through Example 3.1. and Example 3.2.1. Then, pharmacokinetics thereof was measured. DBCOPEG20 kDa-scFVIII G4 B3 Az, DBCOPEG30 kDa-scFVIII G4 B3 Az, and the positive control Advate® were administered by intravenous injection through the tail vein of 13-week-old female hemophilia mice (B6; 129S4-F8tm1Kaz/J (FVIII-knock-out)). Then, activity of FVIII remaining in the blood was measured up to 72 hours. DBCOPEG20 kDa-scFVIII G4 B3 Az, DBCOPEG30 kDa-scFVIII G4 B3 Az, and Advate® were respectively administered through intravenous tail vein injection at a dose of 200 IU/kg; and blood was collected from the retro-orbital venous sinus of each mouse at fixed time points (2 min, 6 hr, 16 hr, 30 hr, 40 hr, 48 hr, 64 hr, 72 hr). To the collected blood was added an anticoagulant (3.2% sodium citrate) so that the anticoagulant had a final concentration of 10% (v/v). Then, centrifugation was performed to obtain plasma. Quantification of the administered substance and quantification of activity of FVIII remaining in the plasma were performed by the CS measurement method as described in Example 1.3. Activity of FVIII remaining in the plasma, which was collected at the respective time points from the mice in each sample group, is illustrated in FIG. 8. In addition, based on the measurement values of FVIII activity remaining in the plasma, PK parameters were derived therefrom through the non-compartmental analysis (NCA) method using WinNonlin software, and the results are shown in Table 4. The half-life of each of DBCOPEG20 kDa-scFVIII G4 B3 Az and DBCOPEG30 kDa-scFVIII G4 B3 Az was calculated from the initial 2 minutes to 72 hours based on the average FVIII activity of mice. As compared with Advate®, the half-life was increased 2.8 times (for DBCOPEG20 kDa-scFVIII G4 B3 Az) and 3.2 times (for DBCOPEG30 kDa-scFVIII G4 B3 Az), respectively. As compared with Advate®, an area under the curve (AUC_{INF}) was also increased by 3.7 times (for DBCOPEG20 kDa-scFVIII G4 B3 Az) and 5.1 times (for DBCOPEG30 kDa-scFVIII G4 B3 Az), respectively. Accordingly, it was identified that in case of being administered by intravenous injection, the modified Factor VIII protein prepared according to the present invention remained in the body for a long time as compared with the existing FVIII, and exhibits a long-acting effect correspondingly.

TABLE 4

| | |
|---|---|
| DBCOPEG20 | DBCOPEG30 |

| Pharmacokinetic | | kDa-seFVIII | kDa-seFVIII |
|---|---|---|---|
| parameters | Advate ® | G4 B3 Az | G4 B3 Az |
| T_{1/2}, terminal (hr) | 7.6 ± 0.8 | 21.1 ± 5.5 | 25.6 ± 1.9 |
| Cₘₐₓ (IU/mL) | 3.9 ± 2.1 | 4.3 ± 0.0 | 5.7 ± 0.3 |
| AUCₗₐₛₜ (hr*IU/mL) | 34.0 ± 2.2 | 120.3 ± 12.2 | 153.7 ± 11.7 |
| AUC_{INF}_obs (hr*IU/mL) | 35.0 ± 2.4 | 131.1 ± 17.4 | 178.9 ± 17.6 |
| AUC %__{Extrap} (%) | 2.8 ± 0.9 | 7.9 ± 3.0 | 9.4 ± 1.7 |
| CL (mL/hr/kg) | 3.6 ± 0.3 | 1.0 ± 0.1 | 0.7 ± 0.1 |
| MRT_{INF} (hr) | 9.6 ± 1.1 | 28.7 ± 2.8 | 31.7 ± 2.9 |

### Example 4.2.1. Pharmacokinetics after Administration by Subcutaneous Injection

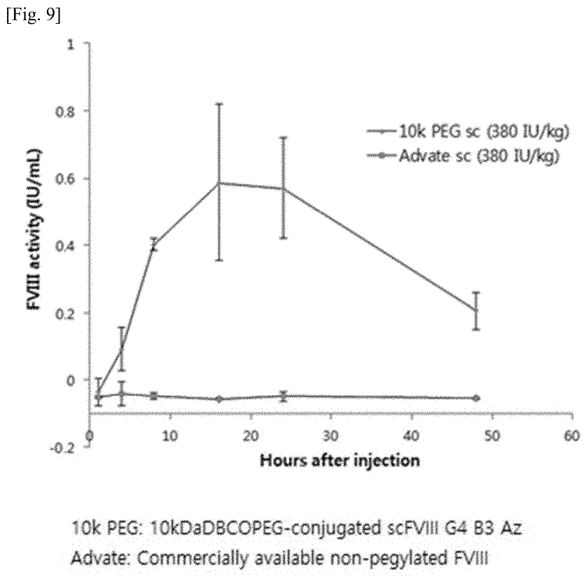

A pharmacokinetic test was performed in which hemophilia mice are administered by subcutaneous injection of glycoPEGylated scFVIIIs produced through Example 3.1. and Example 3.2.1. 13-Week-old female hemophilia mice (B6; 129S4-F8tm1Kaz/J (FVIII-knock-out)) were administered by subcutaneous injection of DBCOPEG10 kDa-scFVIII G4 B3 Az, which is glycoPEGylated scFVIII, and the positive control Advate®. Then, activity of FVIII remaining in the blood was measured up to 48 hours. The mice were administered by subcutaneous injection of each sample at a dose of 380 IU/kg based on FVIII activity (based on the titer of an international titer standard for FVIII); and blood was collected from the retro-orbital venous sinus of each mouse at fixed time points (1 hr, 4 hr, 8 hr, 16 hr, 24 hr, 48 hr). To the collected blood was added an anticoagulant (3.2% sodium citrate) so that the anticoagulant has a final concentration of 10% (v/v). Then, centrifugation was performed to obtain plasma, and the plasma was stored frozen until measurement of FVIII activity. Activity of FVIII remaining in the plasma, which was collected at the respective time points from the mice in each sample group, is illustrated in FIG. 9. Based on the measurement values of FVIII activity remaining in the plasma, PK parameters were derived therefrom through the non-compartmental analysis (NCA) method using WinNonlin software, and the results are shown in Table 5 (pharmacokinetic parameters obtained in a case where DBCOPEG10 kDa-scFVIII G4 B3 Az is subcutaneously administered). DBCOPEG10 kDa-scFVIII G4 B3 Az in each mouse had a terminal half-life which corresponds to a level of 14 to 24 hours, and this was a level that is increased 1.8 to 3.2 times as compared with the half-life of Advate® in Example 4.1. Consistent to the fact that bioavailability of the existing FVIII is less than 1% in a case of being subcutaneously administered in mice (American society of hematology annual meeting (ASH) 2015, Recombinant FVIIIFc-VWF-XTEN demonstrates significant bioavailability following subcutaneous administration in hemophilia A mice), Advate® was present at a level that is not observed in the blood in case of being subcutaneously administered. However, DBCOPEG10 kDa-scFVIII G4 B3 Az was observed, up to 48 hours, at a level that is high enough to cause a hemostatic reaction to bleeding in hemophilia mice. This means that DBCOPEG10 kDa-scFVIII G4 B3 Az, which is glycoPEGylated scFVIII, is stable in a subcutaneous environment and stable even after entering the bloodstream, thereby exhibiting bioavailability higher than the existing FVIII. As a result, it was found that DBCOPEG10 kDa-scFVIII G4 B3 Az, which is glycoPEGylated scFVIII, could be developed as a subcutaneous formulation, unlike the existing FVIII.

TABLE 5

| T_{1/2} | Tₘₐₓ | Cₘₐₓ | AUCₗₐₛₜ | AUC_{INF}_obs |
|---|---|---|---|---|
| (hr) | (hr) | (IU/mL) | (hr*IU/mL) | (hr*IU/mL) |
| 19.2 | 18.7 | 0.618 | 18.1 | 21.7 |

### Example 4.2.2. Pharmacokinetics after Administration by Subcutaneous Injection

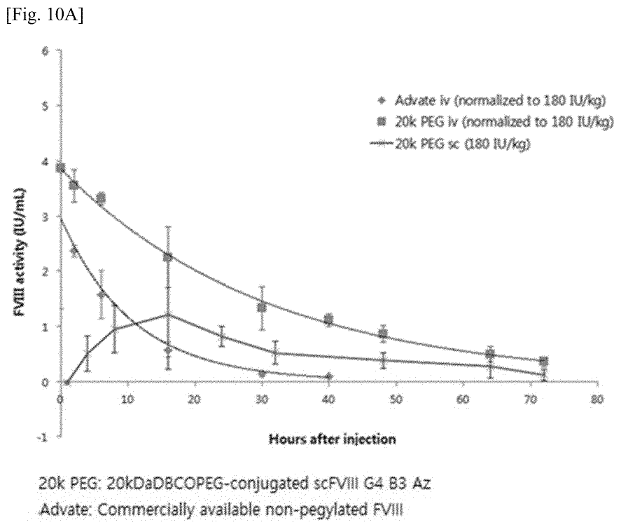

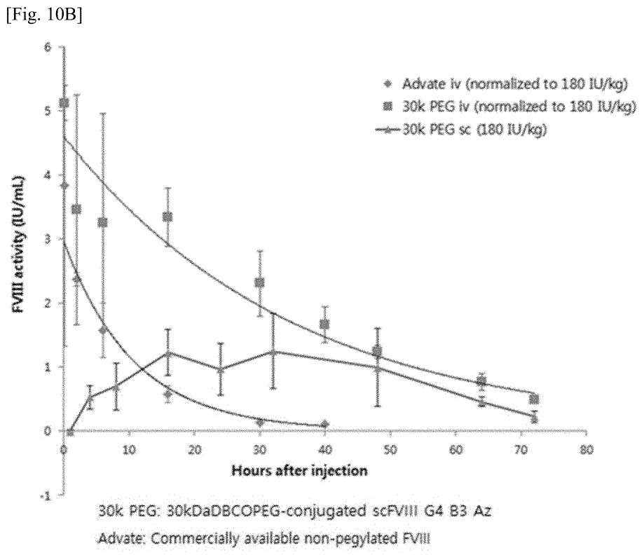

Hemophilia mice were administered by intravenous injection of glycoPEGylated scFVIII produced through Example 3.1. and Example 3.2.1. Then, pharmacokinetics thereof was measured. 13-Week-old female hemophilia mice (B6; 129S4-F8tm1Kaz/J (FVIII-knock-out)) were administered by subcutaneous injection of DBCOPEG20 kDa-scFVIII G4 B3 Az and DBCOPEG30 kDa-scFVIII G4 B3 Az respectively, which are glycoPEGylated scFVIIIs. Then, activity of FVIII remaining in the blood was measured at respective time points. The mice were administered by subcutaneous injection of DBCOPEG20 kDa-scFVIII G4 B3 Az and DBCOPEG30 kDa-scFVIII G4 B3 Az at a dose of 180 IU/kg based on FVIII activity; and blood was collected from the retro-orbital venous sinus of each mouse at fixed time points (1 hr, 4 hr, 8 hr, 16 hr, 24 hr, 48 hr, 72 hr). To the collected blood was added an anticoagulant (3.2% sodium citrate) so that the anticoagulant had a final concentration of 10% (v/v). Then, centrifugation was performed to obtain plasma, and the plasma was stored frozen until measurement of FVIII activity. Quantification of the administered substance and quantification of activity of FVIII remaining in the plasma were performed by the CS measurement method as described in Example 1.3. In order to calculate bioavailability obtained upon subcutaneous administration, in Example 4.1, the AUC obtained upon intravenous administration of DBCOPEG20 kDa-scFVIII G4 B3 Az and DBCOPEG30 kDa-scFVIII G4 B3 Az at a dose of 200 IU/kg was calibrated with 180 IU/kg, which is a dose for subcutaneous administration, so that the bioavailability was calculated. The bioavailability calculated by this calibration is expressed as follows: (AUC_{sc}/AUCᵢᵥ)×(doseᵢᵥ/dose_{sc}) (AUC_{sc}: AUC obtained upon subcutaneous administration; AUCᵢᵥ: AUC obtained upon intravenous administration; doseᵢᵥ: dose for intravenous administration; dose_{sc}: dose for subcutaneous administration). For FVIII and PEG conjugates of FVIII, a reduction rate in vivo in the blood depends only on the substance's concentration, which makes it possible to predict the blood concentration obtained upon intravenous administration with calibration of their initial concentration (J Thromb Haemost 2017 15(6): 1106-1114 Population pharmacokinetics of recombinant coagulation factor VIII-SingleChain in patients with severe hemophilia A; ISTH 2017 pharmacokinetics of N8-GP (turoctocog alfa pegol) dosed subcutaneously to non-human primates and prediction of human PK profile). Activity of FVIII remaining in the plasma, which was collected at respective time points from the mouse group subcutaneously administered the sample, is illustrated in FIG. 10. Based on the average values of FVIII activity in the plasma of each mouse group, PK parameters were derived therefrom through the non-compartmental analysis (NCA) method using WinNonlin software, and the results are shown in Table 6. DBCOPEG20 kDa-scFVIII G4 B3 Az and DBCOPEG30 kDa-scFVIII G4 B3 Az had a terminal half-life of 14.6 hours and 16.9 hours, respectively. The area under the curve (AUC) of Advate® obtained upon subcutaneous administration was less than 1%, whereas DBCOPEG20 kDa-scFVIII G4 B3 Az and DBCOPEG30 kDa-scFVIII G4 B3 Az had bioavailability of 35% and 43%, respectively, in case of being administered by subcutaneous injection. That is, it was found that the glycoPEGylated scFVIII's according to the present invention had bioavailability remarkably higher than that of the existing FVIII (Advate®), thereby showing that the glycoPEGylated scFVIII's, DBCOPEG20 kDa-scFVIII G4 B3 Az and DBCOPEG30 kDa-scFVIII G4 B3 Az, could be developed as a subcutaneous formulation, unlike the existing FVIII (Advate®).

Thus, it is inferred that DBCOPEG20 kDa-scFVIII G4 B3 Az and DBCOPEG30 kDa-scFVIII G4 B3 Az produced by the method of the present invention have high bioavailability in case of being subcutaneously administered for the following reason: in the subcutaneous environment, they are more stable than the existing FVIIII and more completely protected from proteolytic enzymes, and thus have efficient resistance, so that their bioavailability is increased.

TABLE 6

| | DBCOPEG20 | DBCOPEG30 |
|---|---|---|
| | kDa-scFVIII | kDa-scFVIII |
| | G4 B3 Az | G4 B3 Az |
| T_{1/2,terminal} (hr) | 14.6 ± 4.4 | 16.9 ± 10.2 |
| Cₘₐₓ (IU/mL) | 1.6 ± 0.5 | 1.6 ± 0.2 |
| AUCₗₐₛₜ (hr*IU/mL) | 37.9 ± 4.8 | 59.5 ± 14.5 |
| AUC_{INF}_obs (hr*IU/mL) | 40.9 ± 3.1 | 65.9 ± 13.0 |
| *Bioavailability (%) | 35 | 43 |

*(AUC_{sc}/AUCᵢᵥ) × (doseᵢᵥ/dose_{sc})

From these results, it was identified that scFVIII modified with polyethylene glycol polymer by the method according to the present invention could remain longer in the body, that is, in blood and subcutaneous tissue, than Advate®, which is the existing FVIII, so that therapeutic efficacy of a drug can be maintained longer.

As described above, the present invention has been described with reference to an embodiment thereof. However, it will be appreciated by those skilled in the art that the present invention may be variously modified and changed by addition, alteration, deletion, or the like of constituent components without departing from the spirit of the present invention described in the claims, and such modifications or changes are also included within the scope of the present invention.

1. An in vivo long-acting recombinant coagulation factor VIII, comprising a linker which is linked to a sialic acid derivative at the terminus of at least one sugar chain of a coagulation factor VIII, wherein the linker is represented by Formula 1: 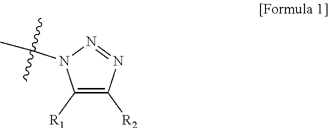
where R₁ and R₂ are each independently C₁₋₈ alkyl, C₆₋₁₂ aryl, C₃₋₆ cycloalkyl, 3- to 10-membered heterocycloalkyl, amino, (amino)C₁₋₆ alkyl, (amino)C₃₋₆ cycloalkyl, (amino)C₆₋₁₂ aryl, (amino)(3- to 10-membered heterocycloalkyl), or 5- to 12-membered heteroaryl (in which the heterocycloalkyl and the heteroaryl may each independently contain at least one heteroatom selected from the group consisting of N, S and O, and may each independently be substituted with at least one substituent selected from the group consisting of halogen, C₁₋₄ alkyl, and CF₃; and the amino may be substituted at least one substituent selected from the group consisting of H, halogen, CN, C₁₋₆ alkyl, haloC₁₋₃ alkyl or C₁₋₆ alkoxycarbonyl), or R₁ and R₂ may be combined with each other to form 8- to 16-membered heterofused ring (in which the heterofused ring may each independently contain at least one heteroatom selected from the group consisting of N, S and O, and may each independently be substituted with at least one substituent selected from the group consisting of halogen, C₁₋₄ alkyl, and CF₃); and
the linker contains a biocompatible polymer connected to at least one of R₁, R₂, or a ring formed when R₁ and R₂ are combined with each other.

2. The in vivo long-acting recombinant coagulation factor VIII of claim 1, wherein the biocompatible polymer is at least one selected from the group consisting of polyethylene glycol (PEG), polysialic acid (PSA), polyoxazoline, heparosan, and combinations thereof.

3. The in vivo long-acting recombinant coagulation factor VIII of claim 2, wherein the polyethylene glycol has a weight average molecular weight of 5 to 40 kDa.

4. The in vivo long-acting recombinant coagulation factor VIII of claim 1, wherein the structure of Formula 1 is at least one selected from the group consisting of Formulas 2 to 18 and isomers thereof: 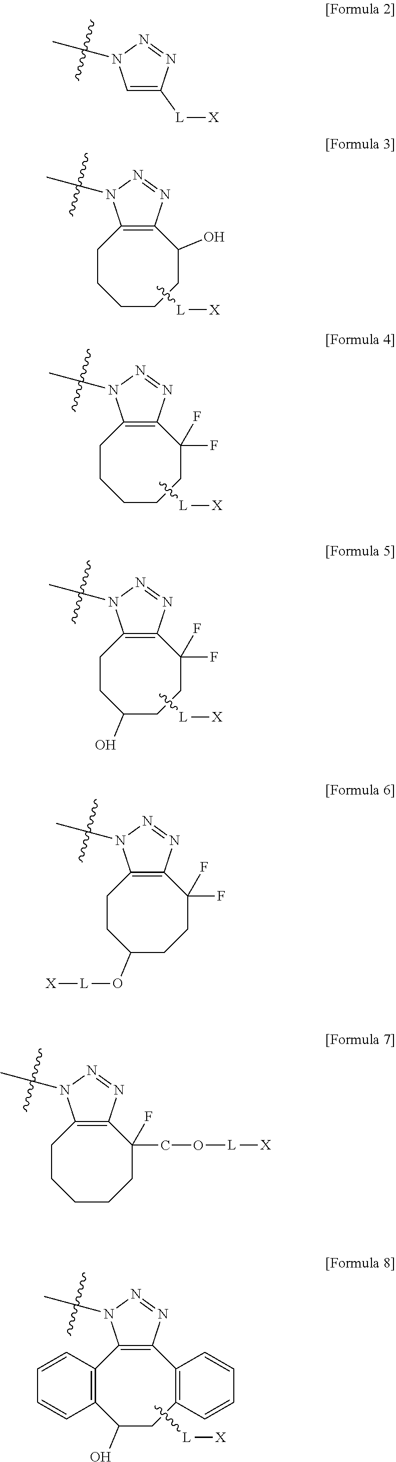 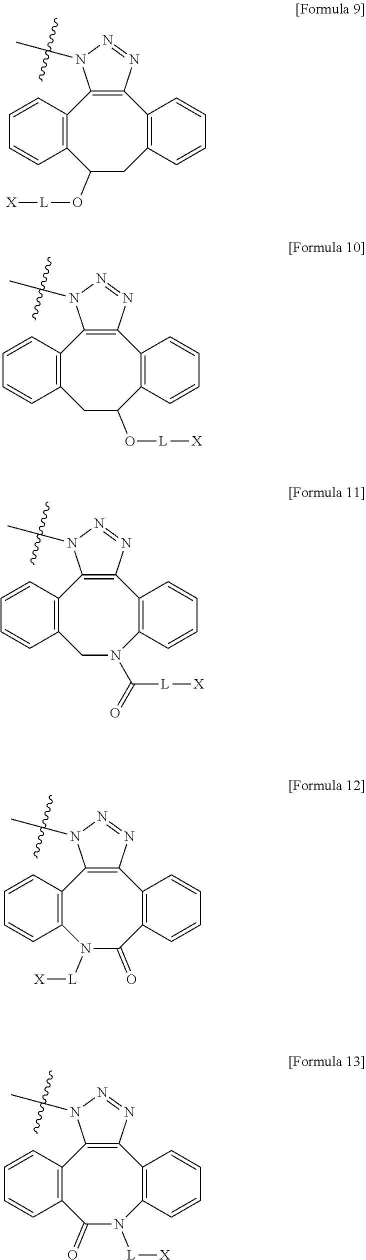 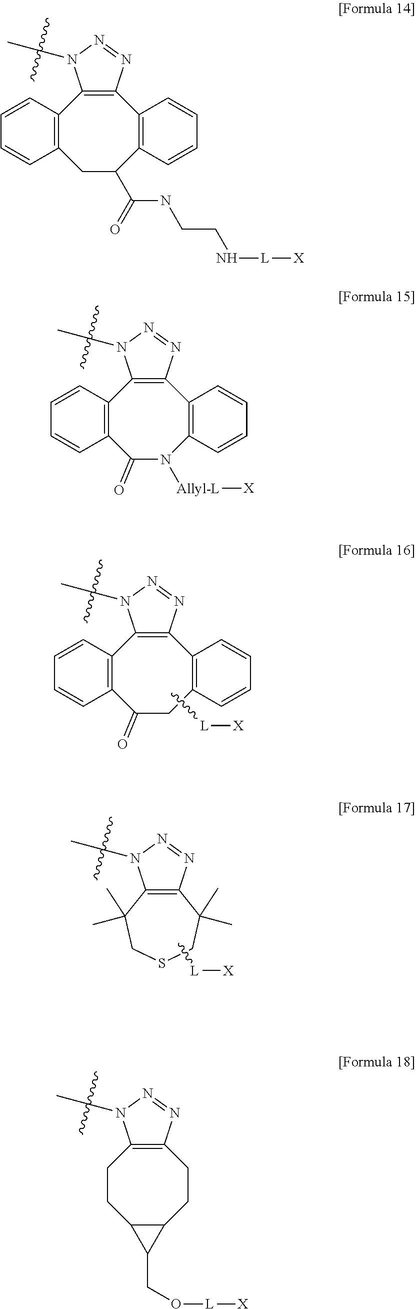
where L may be a single bond, halogen, C₁₋₈ alkyl, C₆₋₁₂ aryl, —CO—, —R₃CONR₃—, —OCONR₃—, or —R₃NCOR₃— (in which R₃ may each independently be selected from H, C₁₋₄ alkyl and C₆₋₁₂ aryl), and X is a biocompatible polymer.

5. The in vivo long-acting recombinant coagulation factor VIII of claim 1, wherein the structure of Formula 1 is at least one selected from the group consisting of Formulas 19 to 26: 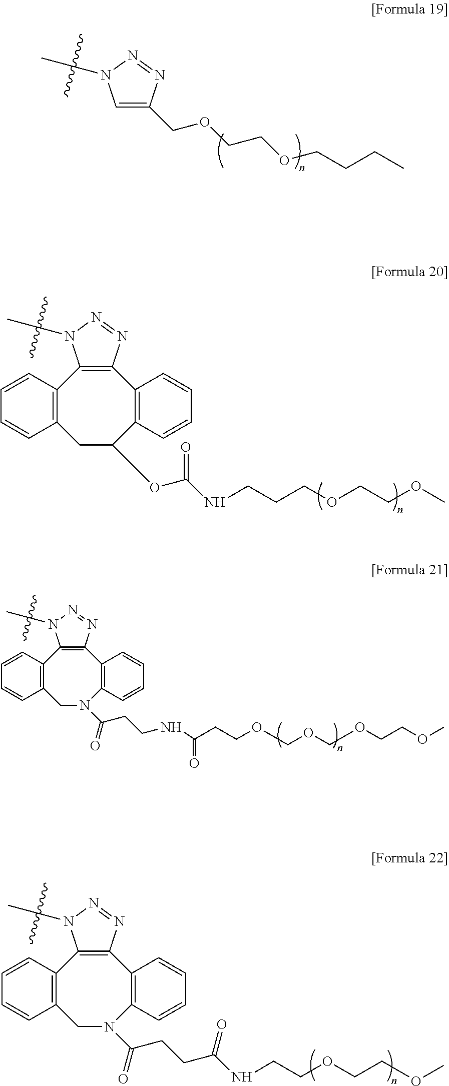 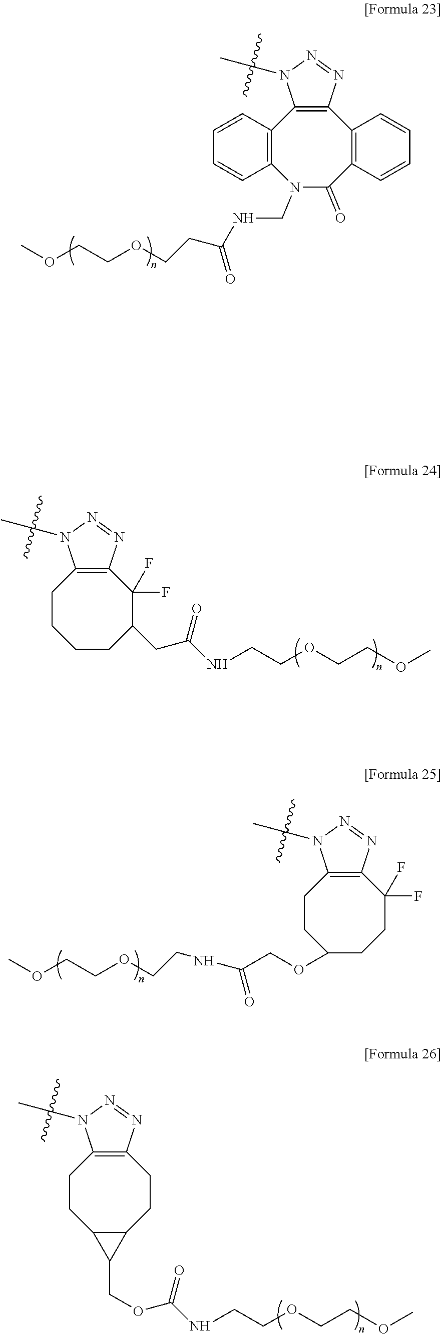
where n is an integer of 100 to 3,000.

6. A method for preparing an in vivo long-acting recombinant coagulation factor VIII, comprising the steps of:
(1) introducing, into a host cell, an expression vector that contains nucleotides encoding a coagulation factor VIII; and
(2) culturing the host cell in a culture medium supplemented with a derivative of a sialic acid metabolite having a first functional group attached thereto, to obtain a coagulation factor VIII to which the sialic acid derivative having the first functional group attached thereto is introduced at the terminus of at least one sugar chain.

7. The method of claim 6, wherein the first functional group is an azide group or an alkyne group.

8. The method of claim 6, wherein in step (2), the derivative of the sialic acid metabolite is at least one compound selected from the group consisting of Formulas 27 to 32 and isomers thereof:

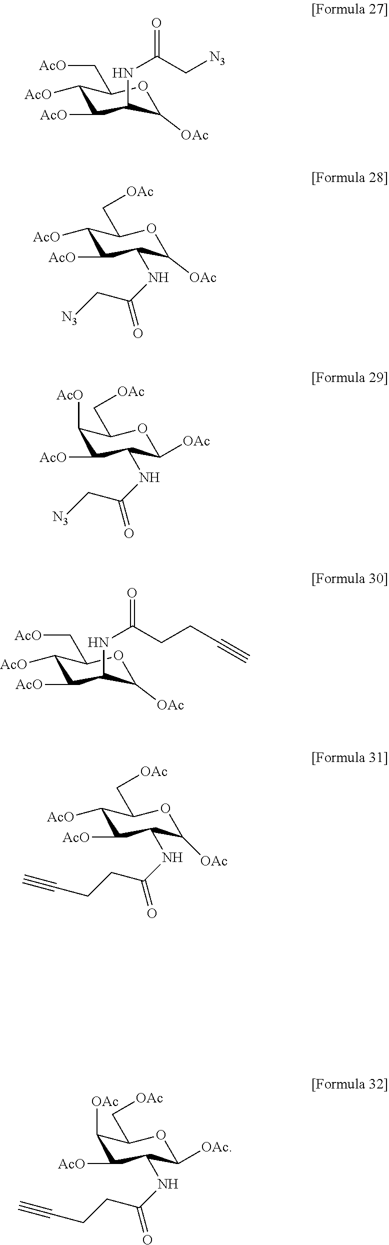

9. The method of claim 6, wherein in step (2), the derivative of the sialic acid metabolite is supplemented in a concentration of 0.1 to 300 uM when the host cell has a concentration of 1.0×10⁵ or higher.

10. The method of claim 9, wherein in step (2), the temperature is adjusted to 29° C. to 35° C. when the derivative of the sialic acid metabolite is supplemented.

11. The method of claim 6, further comprising the steps of:
(3) removing the derivative of the sialic acid metabolite from the culture medium;
(4) adding, to the culture medium, a compound having a second functional group and a biocompatible polymer attached thereto, to allow a click reaction; and
(5) collecting a coagulation factor VIII to which the biocompatible polymer is attached at the terminus of at least one sugar chain.

12. The method of claim 11, wherein the biocompatible polymer is at least one selected from the group consisting of polyethylene glycol (PEG), polysialic acid (PSA), polyoxazoline, heparosan, and combinations thereof.

13. The method of claim 11, wherein the second functional group is an alkyne group or a cycloalkyne group when the first functional group is an azide group; and the second functional group is an azide group when the first functional group is an alkyne group.

14. The method of claim 11, wherein in step (4), the compound having the second functional group and the biocompatible polymer attached thereto is at least one compound selected from the group consisting of Formulas 33 to 49 and isomers thereof: 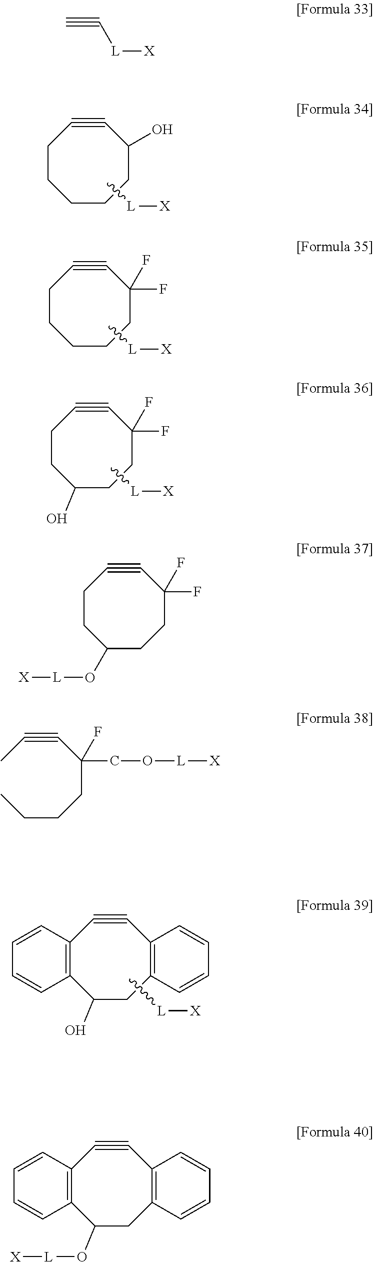 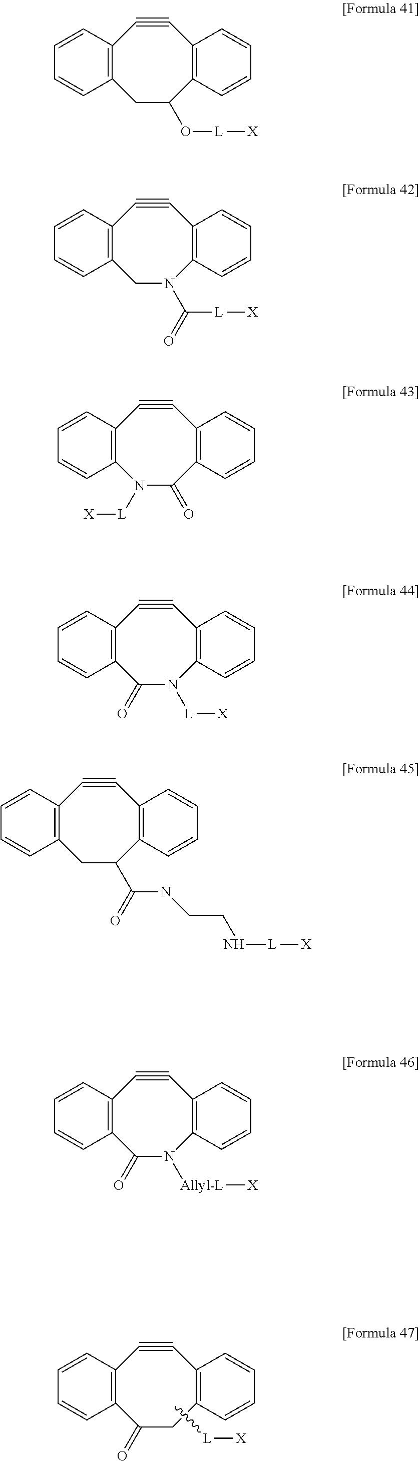 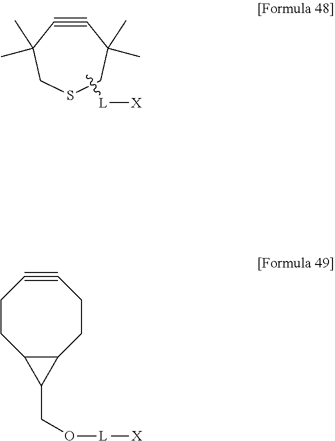
where L may be a single bond, halogen, C₁₋₈ alkyl, C₆₋₁₂ aryl, —CO—, —R₃CONR₃—, —OCONR₃—, or —R₃NCOR₃— (in which R₃ may each independently be selected from H, C₁₋₄ alkyl and C₆₋₁₂ aryl), and X is a biocompatible polymer.

15. The method of claim 6, wherein the in vivo long-acting recombinant coagulation factor VIII comprises a linker which is linked to the sialic acid derivative at the terminus of at least one sugar chain of the coagulation factor VIII, wherein the linker is represented by Formula 1: 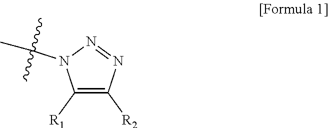
where R₁ and R₂ are each independently C₁₋₈ alkyl, C₆₋₁₂ aryl, C₃₋₆ cycloalkyl, 3- to 10-membered heterocycloalkyl, amino, (amino)C₁₋₆ alkyl, (amino)C₃₋₆ cycloalkyl, (amino)C₆₋₁₂ aryl, (amino)(3- to 10-membered heterocycloalkyl), or 5- to 12-membered heteroaryl (in which the heterocycloalkyl and the heteroaryl may each independently contain at least one heteroatom selected from the group consisting of N, S and O, and may each independently be substituted with at least one substituent selected from the group consisting of halogen, C₁₋₄ alkyl, and CF₃; and the amino may be substituted at least one substituent selected from the group consisting of H, halogen, CN, C₁₋₆ alkyl, haloC₁₋₃ alkyl or C₁₋₆ alkoxycarbonyl), or R₁ and R₂ may be combined with each other to form 8- to 16-membered heterofused ring (in which the heterofused ring may each independently contain at least one heteroatom selected from the group consisting of N, S and O, and may each independently be substituted with at least one substituent selected from the group consisting of halogen, C₁₋₄ alkyl, and CF₃); and
the linker contains a biocompatible polymer connected to at least one of R₁, R₂, or a ring formed when R₁ and R₂ are combined with each other.

16. The method of claim 15, wherein the structure of Formula 1 is at least one selected from the group consisting of Formulas 19 to 26: 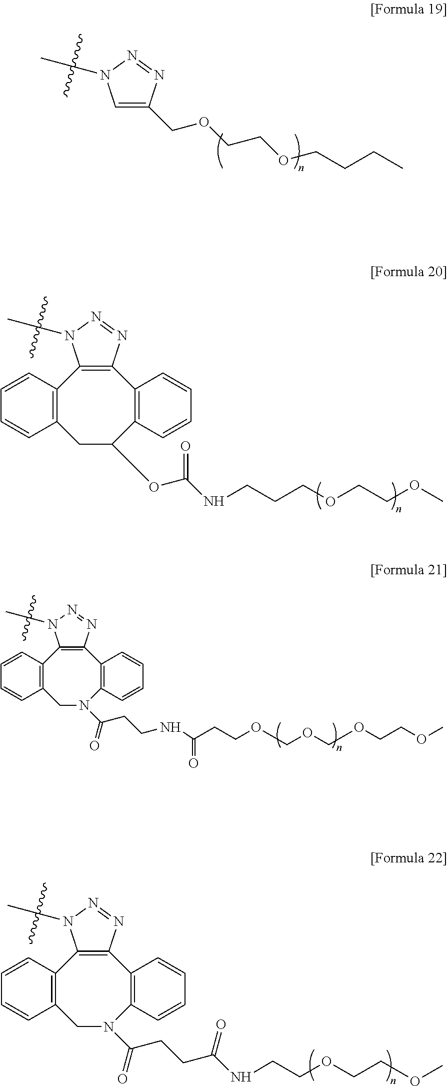 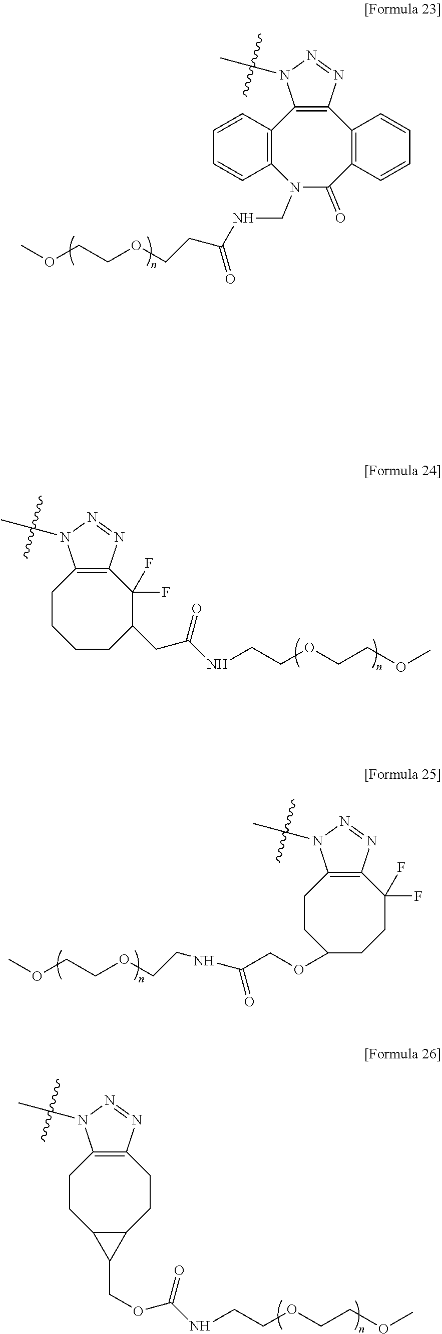
where n is an integer of 100 to 3,000.
